SEQUENCE LISTING (6 sequences)

SEQ ID NO: 1 - DNA, 22 bp, Artificial sequence
  Amplification primer
aggcagctttgtggagatggtg

SEQ ID NO: 2 - DNA, 22 bp, Artificial sequence
  Amplification primer
cgggaaatggaaaggctactcc

SEQ ID NO: 3 - DNA, 21 bp, Artificial sequence
  Amplification primer
tggatgtggaatgtgtgcgag

SEQ ID NO: 4 - protein, 6 aa, Artificial sequence
  Portion of fusion protein
HHHHHH

SEQ ID NO: 5 - protein, 42 aa, Homo sapiens [sequence omitted]

SEQ ID NO: 6 - protein, 42 aa, Mouse
DAEFGHDSGFEVRHQKLVFFAEDVGSNKGAIIGLMVGGVVIA

(19) United States
(12) Patent Application Publication
(10) Pub. No.: US 2004/0132680 A1
(43) Pub. Date: Jul. 8, 2004

(54) Beta secretase transgenic organisms and methods of use thereof

(75) Inventors: Philip C. Wong, Timonium, MD (US); Huaibin Cai, Baltimore, MD (US); Donald L. Price, Columbia, MD (US)

Correspondence Address: Lisa A. Haile, J.D., Ph.D., GRAY CARY WARE & FREIDENRICH LLP, Suite 1100, 4365 Executive Drive, San Diego, CA, 92121-2133, US

(73) Assignee: THE JOHNS HOPKINS UNIVERSITY SCHOOL OF MEDICINE

(21) Appl. No.: 10/656,624

(22) Filed: Sep. 4, 2003

Related U.S. Application Data
(62) Division of application No. 09/708,096, filed on Nov. 3, 2000, now abandoned.
(60) Provisional application No. 60/244,051, filed on Oct. 27, 2000.

(51) Int. Cl. A61K 48/00; A61K 39/395

(52) U.S. Cl. 514/44; 424/155.1

(57) ABSTRACT

The invention provides transgenic non-human animals, such as transgenic rodents and transgenic non-human mammalian cells harboring a transgene which eliminates the expression of a β-secretase BACE1. Also provided are methods of diagnosing neurodegenerative diseases including Alzheimer's Disease as well as methods of identifying agents which modulate or treat Alzheimer's Disease and related pathology.

### CROSS REFERENCE TO RELATED APPLICATIONS

This application claims priority under 35 U.S.C. §119(e)(1) from Provisional Application Serial No. ______, filed Oct. 27, 2000, entitled “Beta-Secretase (BACE1) Knockout Mice”.

### STATEMENT AS TO FEDERALLY SPONSORED RESEARCH

This invention resulted from research funded in whole or part by the National Institutes of Health, Grant No. 1P01 AG14248, and 2P50 AG05146. The Federal Government may have certain rights in this patent.

### FIELD OF THE INVENTION

The invention generally relates to neurological diseases and more specifically to Alzheimer's disease and BACE1 biochemistry.

### BACKGROUND

The amyloidoses are a group of pathological conditions in which normally soluble proteins polymerize to form insoluble amyloid fibrils and amyloid deposits. More than 15 proteins form amyloid fibrils resulting in diverse clinical conditions. Amyloidoses are usually classified into systemic amyloidoses and localized amyloidoses. Major systemic amyloidoses include AL amyloidosis, amyloid A amyloidosis, and familial transthyretin amyloidosis; the corresponding amyloid proteins in these amyloidoses are AL amyloid, amyloid A protein, and transthyretin, respectively. Prominent localized amyloidoses include Alzheimer's disease, prion diseases, and type II diabetes; the corresponding amyloid proteins in these diseases are amyloid β peptide, scrapie prion protein, and human amylin, respectively (Sipe, Annu. Rev. Biochem. 61:947-975, 1992).

Amyloid fibrils, regardless of the amyloid protein from which they are formed, have a cytotoxic effect on various cell types including primary cultured hippocampal neurons (Yankner et al., Science 250:279-282, 1990), pancreatic islet β cells (Lorenzo et al. Nature 368:756-760, 1994) and clonal cell lines (Behl et al., Biochem Biophys. Res. Commun. 186:944-952, 1992; O'Brien et al., Am. J. Pathol. 147:609-616, 1995). In fact, only amyloid proteins in fibrillar form are cytotoxic (Pike et al., Brain Res. 563:311-314, 1991; Lorenzo and Yankner, Proc. Natl. Acad. Sci. 91:12243-12247, 1994). It is likely that the cytotoxic effect of fibrils is mediated by a common mechanism (Lorenzo and Yankner, supra; Schubert et al., Proc. Natl. Acad. Sci. USA 92:1989-1993, 1995). Modulation of amyloid protein aggregation is one means of blocking or reducing amyloid toxicity.

Alzheimer's disease (AD) is a progressive disease known generally as senile dementia. Broadly speaking the disease falls into two categories, namely late onset and early onset. Late onset, which occurs in old age (65+ years), may be caused by the natural atrophy of the brain occurring at a faster rate and to a more severe degree than normal. Early onset AD is much more infrequent but shows a pathologically identical dementia with brain atrophy which develops well before the senile period, e.g., between the ages of 35 and 60 years.

Alzheimer's disease is characterized by the presence of numerous amyloid plaques and neurofibrillary tangles (highly insoluble protein aggregates) present in the brains of AD patients, particularly in those regions involved with memory and cognition. In particular, it has been discovered that the production of β-amyloid peptide, a major constituent of the amyloid plaque, can result from mutations in the gene encoding amyloid precursor protein, a protein which when normally processed will not produce the β-amyloid peptide. It is presently believed that a normal (non-pathogenic) processing of the β-amyloid precursor protein occurs via cleavage by a putative “α-secretase” which cleaves between amino acids 16 and 17 of the protein. It is further believed that pathogenic processing occurs via a putative “β-secretase” at the amino-terminus of the β-amyloid peptide within the precursor protein. Moreover, β-amyloid peptide appears to be toxic to brain neurons, and neuronal cell death is associated with the disease.

β-amyloid peptide (also referred to as A4, βAP, Aβ, or AβP; see, U.S. Pat. No. 4,666,829 and Glenner and Wong (1984) Biochem. Biophys. Res. Commun. 120: 1131) is derived from β-amyloid precursor protein (PAPP), which is expressed in differently spliced forms of 695, 751, and 770 amino acids. See, Kang et al., Nature 325:773, 1987; Ponte et al., Nature 331:525, 1988; and Kitaguchi et al., Nature 331:530, 1988. Normal processing of amyloid precursor protein (APP) involves proteolytic cleavage at a site between residues Lys¹⁶ and Leu¹⁷ (as numbered where Asp⁵⁹⁷ is residue 1 in Kang et al. (1987), supra), near the transmembrane domain, resulting in the constitutive secretion of an extracellular domain which retains the remaining portion of the β-amyloid peptide sequence (Esch et al., Science 248:1122-1124, 1990). This pathway appears to be widely conserved among species and present in many cell types. See, Weidemann et al., Cell 57:115-126, 1989; and Oltersdorf et al., J. Biol. Chem. 265:4492-4497, 1990. This normal pathway cleaves within the region of the precursor protein which corresponds to the β-amyloid peptide, thus apparently precluding its formation. Another constitutively secreted form of βAPP has been noted (Robakis et al. Soc. Neurosci. Oct. 26, 1993, Abstract No. 15.4, Anaheim, Calif.) which contains more of the βAPP sequence carboxy terminal to that form described by Esch et al. supra.

Reports show that soluble β-amyloid peptide is produced by healthy cells into culture media (Haass et al., Nature 359:322-325, 1992) and in human and animal CSF (Seubert et al., Nature 359:325-327, 1992). Palmert et al., Biochm. Biophys. Res. Comm. 165:182-188, 1989, describes three possible cleavage mechanisms for PAPP and presents evidence that PAPP cleavage does not occur at methionine⁵⁹⁶ in the production of soluble derivatives of βAPP. U.S. Pat. No. 5,200,339, discusses the existence of certain proteolytic factor(s) which are putatively capable of cleaving βAPP at a site near the PAPP amino-terminus.

The APP gene is known to be located on human chromosome 21. A locus segregating with familial Alzheimer's disease has been mapped to chromosome 21 (St. George Hyslop et al., Science 235:885, 1987) close to the APP gene. Recombinants between the APP gene and the AD locus have been previously reported (Schellenberg et al., Science 241:1507, 1988; Schellenberg et al., Am. J. Hum. Genetics 48:563, 1991; Schellenberg et al., Am. J. Hum. Genetics 49:511, 1991).

The identification of mutations in the amyloid precursor protein gene which cause familial, early onset Alzheimer's disease is evidence that amyloid metabolism is the central event in the pathogenic process underlying the disease. Four reported disease-causing mutations include with respect to the 770 isoform, V7171 (Goate et al., Nature 349:704, 1991), V717G (Chartier Harlan et al., Nature 353: 844, 1991), V717F (Murrell et al., Science 254:97, 1991) and with respect to the 695 isoform, a double mutation changing K595N and M596L (Mullan et al., Nature Genet 1:345, 1992; Citron et al., Nature 360:672, 1992) referred to as the Swedish mutation.

The development of experimental models of Alzheimer's disease that can be used to further study the underlying biochemical events involved in AD pathogenesis would be highly desirable. Such models could presumably be employed, in one application, to screen for agents that alter the degenerative course of Alzheimer's disease. For example, a model system of Alzheimer's disease could be used to screen for environmental factors that induce or accelerate the pathogenesis of AD. In contradistinction, an experimental model could be used to screen for agents that inhibit, prevent, or reverse the progression of AD. Presumably, such models could be employed to develop pharmaceuticals that are effective in preventing, arresting, or reversing AD. It would also be desirable to have a model that can be used as a standard or control for comparison of agents the modulate amyloid deposition or activity.

### SUMMARY OF THE INVENTION

The present invention provides a method for modulating the production of Aβ11-40/42 peptide fragments. The method includes contacting a sample or cell containing a beta-site APP-cleaving enzyme 1 (BACE1) and an amyloid precursor protein (APP) with a BACE1-modulating agent such that production of Aβ11-40/42 is modulated. The contacting can be in vivo or in vitro.

In another embodiment, the invention provides a method for identifying a compound which inhibits beta-site APP-cleaving enzyme 1 (BACE1) expression or activity. The method includes incubating components including the compound, BACE1 polynucleotide or polypeptide, and an amyloid precursor protein (APP) under conditions sufficient to allow the components to interact and measuring the production of a BACE1 specific enzymatic product.

Also provided are methods for diagnosing a subject having or at risk of having an Aβ11-40/42 peptide accumulation disease. The method includes measuring the amount of beta-site APP-cleaving enzyme 1 (BACE1) in a biological sample from the subject; comparing the amount BACE1 with a normal standard value of BACE1, wherein a difference between the measured amount and the normal sample or standard value provides an indication of the diagnosis of Aβ11-40/42. The sample can be, for example, blood, serum, cerebrospinal fluid or central nervous system (CNS) tissue.

In yet another embodiment, the invention provides a method for diagnosing a subject having or at risk of having Alzheimer's Disease, including measuring Aβ11-40/42 in a biological sample from the subject; comparing the amount of Aβ11-40/42 with a normal sample or standard value of Aβ11-40/42, wherein a difference between the amount in the normal sample or standard value is indicative of a subject having or at risk of having Alzheimer's disease.

In another embodiment, the invention provides a transgenic non-human animal having a transgene disrupting expression of BACE1, chromsomally integrated into the germ cells of the animal, and have a phenotype of reduced Aβ peptide as compared with a wild-type animal.

In another embodiment, the invention provides a method for producing a transgenic non-human animal having a phenotype characterized by reduced expression of BACE1 polypeptide. The method includes introducing at least one transgene into a zygote of an animal, the transgene(s) comprising a DNA construct encoding a selectable marker, transplanting the zygote into a pseudopregnant animal, allowing the zygote to develop to term, and identifying at least one transgenic offspring whose genome comprises a disruption of the endogenous BACE1 polynucleotide sequence by the transgene.

In yet another embodiment, the invention provides a method for identifying an agent that modulates the expression or activity of BACE1. The method includes administering an agent to be tested to an organism; and comparing the phenotype of the organism contacted with the agent with that of a BACE1-knockout organism not contacted with the agent, whereby a phenotype substantially equal to the BACE1-knockout organism is indicative of an agent that modulates BACE1 expression or activity.

The invention also provides a method for screening for an agent, which ameliorates symptoms of Alzheimer's disease. The method includes comparing an effect of an agent on an organism contacted with the agent with that of a BACE1-knockout organism not contacted with the agent, wherein the organism has a phenotype associated with Alzheimer's Disease and wherein an agent which ameliorates said phenotype is identified by having a substantially equal or superior phenotype of the organism in comparison with the BACE1-knockout organism.

In yet another embodiment, the invention provides a method for screening for an agent, which ameliorates symptoms of Alzheimer's disease. The method includes comparing an effect of an agent on a transgenic organism contacted with the agent with that of a BACE1-knockout organism not contacted with the agent, wherein the transgenic organism is characterized as having a phenotype of impaired performance on memory learning tests or abnormal neuropathology in a cortico-limbic region of the brain and the BACE1-knockout organism has a phenotype of reduced expression of BACE1, wherein the impaired performance and the abnormal neuropathology are in compared with the BACE1-knockout organism, whereby an agent which ameliorates the symptoms is identified by substantially equal or superior performance of the transgenic organism as compared with the BACE1-knockout organism on the memory and learning tests.

The invention also provides a kit useful for the detection of an Aβ11-40/42 accumulation disorder comprising carrier means containing therein one or more containers wherein a first container contains a nucleic acid probe that hybridizes to a nucleic acid sequence BACE1 or an antibody probe specific for BACE1 or Aβ11-40/42.

In yet another embodiment, the invention provides a method for predicting the therapeutic effectiveness of a compound for treating Alzheimer's disease in a subject by measuring the accumulation of Aβ11-40/42 peptide fragments in the subject or the level of BACE1 polynucleotide or polypeptide before and after treatment with the compound, wherein a decrease in accumulation of peptide fragments or a decrease in the level of BACE1 polynucleotide or polypeptide after treatment is indicative of a compound that is effective in treating the disease.

In another embodiment, the invention provides a method for monitoring the progression of Alzheimer's disease by measuring the accumulation of Aβ11-40/42 peptide fragments in the subject or the level of BACE1 polynucleotide or polypeptide at a first time point and a second time point, thereby monitoring the progression of the disease.

### BRIEF DESCRIPTION OF THE DRAWINGS

FIG. 1A is a map of the wild-type BACE1 locus, the targeting vector, and the disrupted BACE1 allele. The first coding exon of BACE1 is indicated by black box. The targeting vector shows the replacement of the first coding exon and flanking genomic sequences by the neomycin gene (neo) and the HSV thymidine kinase gene (tk). Arrows indicate the sites within the targeted and wild-type alleles from which PCR primers were chosen for genotyping. Lines below denote expected sizes for SacI-digested fragments detected by a 5′-flanking probe (a 0.45 kb HindIII/PstI fragment, black bar) from targeted and endogenous BACE1 alleles. B, BamHI; H, HindIII; P, PstI; S, SacI; X, XbaI.

FIG. 1B shows an analysis of genomic DNA from BACE1^{+/-} crosses by Southern blot. The SacI fragments detected for wild-type (8.0 kb) and targeted (5.4 kb) BACE1 alleles with the 5′ probe are indicated.

FIG. 1C shows PCR analysis of DNA extracted from embryos using primers indicated in A, the 157 bp or 272 bp fragment is specific to the targeted or endogenous BACE1 allele, respectively.

FIG. 1D shows total protein extracts (30 μg) of wild-type (+/+), heterozygous (+/−), and homozygous BACE1 knockout (−/−) from El 6.5 embryos. Embryos were immunoblotted using rabbit polyclonal antisera specific for epitopes in the N terminal 46-163 amino acids of BACE1, and superoxide dismutase 1 (SOD1).

FIG. 2A shows a sequence alignment of Aβ1-42 denoting differences between the human and mouse protein sequences (bolded amino acids). The cleavage sites corresponding to BACE1, a and γ secretases are marked and numbered. The asterisk indicate the start of the transmembrane domain.

FIG. 2B shows IP-MS analysis of secreted AP peptides from primary cultured cortical neurons derived from wide-type (+/+), heterozygous (+/−), and homozygous BACE1 knockout (−/−) E16.5 embryos using the Ciphergen ProteinChip system. Peaks corresponding to mouse Aβ peptides, 17-40, 11-40, 11-42, 1-40 and 1-42 are marked by asterisk. The mass of each peptide is labeled within brackets.

FIG. 2C shows a determination of Aβ1-40 and Aβ1-42 levels from conditioned media of BACE^{+/+} and BACE^{-/-} neuronal cultures following 4 days of infection with adenovirus expressing humanized APPswe by ELISA. The concentrations of Aβ peptides for each genotype are plotted (pg/ml) as mean+/−standard deviation (n=3).

FIG. 2D shows conditioned media from BACE^{+/+} and BACE^{-/-} neuronal cell cultures radiolabeled with ³⁵S-methionine after 4 days of infection with recombinant adenovirus expressing humanized APPswe were immunoprecipated with 4G8, an antisera specific for Aβ peptides.

FIG. 2E shows a detergent lysates from BACE^{+/+} and BACE^{-/-} neuronal cell cultures radiolabeled with ³⁵S-methionine after 4 days of infection with recombinant adenovirus expressing humanized APPswe. The cells were immunoprecipated with CT15, an antisera recognizing APP C terminus. BACE deficient neurons failed to generate APP β-CTF.

FIGS. 3A-D shows a gel from neuronal cultures infected with adenovirus. (A) Following 4 days of infection with adenovirus expressing humanized APPswe, BACE^{+/+} (lanes 1-4) and BACE^{-/-} (lanes 5-8) neuronal cultures were pulse-labeled for 45 minutes (lanes 1 and 5) with ³⁵S-methionine, then chased in the presence of cold L-methionine for 1 hr (lanes 2 and 6), 2 hr (lanes 3 and 7), and 4 hr (lanes 4 and 8). Full-length APP and CTFs of APP were immunoprecipitated with CT15. Aβ and p3 peptides (B), soluble APP derivatives (APP^{s})(C), or α-secretase-generated APP^{s} (APP^{sα}) (D), were immunoprecipitated with 4G8, 22C11, or 6E10 antisera, respectively, from conditioned media of the corresponding neuronal cultures as shown in (A).

FIG. 3E is a quantitative analysis of APP^{sα} release. Experiments were performed in duplicate on different days. The APP^{sα} and APP^{s} signals at each point of the pulse-chase experiments were quantified by phosphoimaging.

### DETAILED DESCRIPTION OF THE INVENTION

Alzheimer's disease, a progressive neurodegenerative disorder causing dementia in the elderly, is characterized by the deposition of Aβ-amyloid and neurofibrillary tangles in a variety of brain region, particularly the hippocampus and cerebral cortex. Endoproteolytic cleavages of APP by β- and γ-secretase activities result in the generation of toxic Aβ peptides. Two homologous β-secretases, termed BACE1 and BACE2, have recently been cloned and shown to be transmembrane aspartyl proteases that cleave APP at the +1 Aβ site. Initial studies indicated that BACE1 and BACE2 mRNA are expressed ubiquitously, although BACE2 is expressed at lower levels in brain.

The present invention is based upon the discovery that BACE1-knockout transgenic organisms lacking normal expression of BACE1 have reduced accumulation of APP peptide fragments. The transgenic organisms have led to the discovery that BACE1 is the α-secretase responsible for the Aβ+11 peptide fragment of APP. Accordingly, the invention includes diagnostic methods and compositions useful of detecting AD as well as other BACE1- and APP-associated disorders. Based on the discovery of the role of BACE1 in AD, the invention now provides screening assays for drugs that inhibit or prevent Aβ11-40/42 production and therefore may be effective for AD treatment.

The term “isolated” means altered “by the hand of man” from its natural state; i.e., if it occurs in nature, it has been changed or removed from its original environment, or both. For example, a naturally occurring polynucleotide or a polypeptide naturally present in a living animal in its natural state is not “isolated”, but the same polynucleotide or polypeptide separated from the coexisting materials of its natural state is “isolated”, as the term is employed herein. As part of or following isolation, a polynucleotide can be joined to other polynucleotides, such as for example DNAs, for mutagenesis studies, to form fusion proteins, and for propagation or expression of the polynucleotide in a host. The isolated polynucleotides, alone or joined to other polynucleotides, such as vectors, can be introduced into host cells, in culture or in whole organisms. Such polynucleotides, when introduced into host cells in culture or in whole organisms, still would be isolated, as the term is used herein, because they would not be in their naturally occurring form or environment. Similarly, the polynucleotides and polypeptides may occur in a composition, such as a media formulation (solutions for introduction of polynucleotides or polypeptides, for example, into cells or compositions or solutions for chemical or enzymatic reactions).

Polynucleotide or nucleic acid sequence refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. In addition, the polynucleotide sequence involved in producing a polypeptide chain can include regions preceding and following the coding region (leader and trailer) as well as intervening sequences (introns) between individual coding segments (exons) depending upon the source of the polynucleotide sequence.

The term polynucleotide(s) generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, a polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

In addition, the polynucleotides or nucleic acid sequences may contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

Nucleic acid sequences can be created which encode a fusion protein and can be operatively linked to expression control sequences. “Operatively linked” refers to a juxtaposition wherein the components so described are in a relationship permitting them to function in their intended manner. For example, a coding sequence is “operably linked” to another coding sequence when RNA polymerase will transcribe the two coding sequences into a single mRNA, which is then translated into a single polypeptide having amino acids derived from both coding sequences. The coding sequences need not be contiguous to one another so long as the expressed sequences ultimately process to produce the desired protein. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the of the polynucleotide sequence. Both constitutive and inducible promoters, are included in the invention (see e.g., Bitter et al., Methods in Enzymology 153:516-544, 1987). For example, when cloning in bacterial systems, inducible promoters such as pL of bacteriophage, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention.

A nucleic acid sequence of the invention including, for example, a polynucleotide encoding a fusion protein, may be inserted into a recombinant expression vector. A recombinant expression vector generally refers to a plasmid, virus or other vehicle known in the art that has been manipulated by insertion or incorporation of a nucleic acid sequences. For example, a recombinant expression vector of the invention includes a polynucleotide sequence encoding a polypeptide having BACE1 activity or a fragment thereof or encoding an APP fusion product or fragment thereof. The expression vector typically contains an origin of replication, a promoter, as well as specific genes which allow phenotypic selection of the transformed cells. Vectors suitable for use in the invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988), baculovirus-derived vectors for expression in insect cells, cauliflower mosaic virus, CaMV; tobacco mosaic virus, TMV. The nucleic acid sequences of the invention can also include a localization sequence to direct the indicator to particular cellular sites by fusion to appropriate organellar targeting signals or localized host proteins. For example, a polynucleotide encoding a localization sequence, or signal sequence, can be used as a repressor and thus can be ligated or fused at the 5′ terminus of a polynucleotide encoding a polypeptide of the invention such that the localization or signal peptide is located at the amino terminal end of a resulting polynucleotide/polypeptide. The construction of expression vectors and the expression of genes in transfected cells involves the use of molecular cloning techniques also well known in the art. (See, for example, Sambrook et al., Molecular Cloning—A Laboratory Manual, Cold Spring Harbor Laboratory, Cold Spring Harbor, N.Y., 1989, and Current Protocols in Molecular Biology, M. Ausubel et al., eds., (Current Protocols, a joint venture between Greene Publishing Associates, Inc. and John Wiley & Sons, Inc., most recent Supplement)). These methods include in vitro recombinant DNA techniques, synthetic techniques and in vivo recombination/genetic recombination. (See also, Maniatis, et al., Molecular Cloning A Laboratory Manual, Cold Spring Harbor Laboratory, N.Y., 1989).

In yeast, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; Grant, et al., “Expression and Secretion Vectors for Yeast,” in Methods in Enzymology, Eds. Wu & Grossman, 1987, Acad. Press, N.Y., Vol. 153, pp.516-544, 1987; Glover, DNA Cloning, Vol. 11, IRL Press, Wash., D.C., Ch. 3, 1986; and Bitter, “Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

An alternative expression system which could be used to express a BACE (e.g., BACE1) polypeptide of the invention is an insect system. In one such system, Autographa californica nuclear polyhedrosis virus (AcNPV) is used as a vector to express foreign or mutated polynucleotide sequences. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed, see Smith, et al., J. Viol. 46:584, 1983; Smith, U.S. Pat. No. 4,215,051.

The vectors of the invention can be used to transform a host cell. By transform or transformation is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

A transformed cell or host cell generally refers to a cell (e.g., prokaryotic or eukaryotic) into which (or into an ancestor of which) has been introduced, by means of recombinant DNA techniques, a DNA molecule encoding an APP or BACE polypeptide or a fragment thereof.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, methods of transfection or transformation with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with DNA sequences encoding a BACE1 polypeptide and a second foreign DNA molecule encoding APP, or a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Typically, a eukaryotic host will be utilized as the host cell. The eukaryotic cell may be a yeast cell (e.g., Saccharomyces cerevisiae), an insect cell (e.g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and WI38.

Mammalian cell systems which utilize recombinant viruses or viral elements to direct expression may be engineered. For example, when using adenovirus expression vectors, a polynucleotide encoding a BACE (e.g., BACE1) polypeptide may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a BACE polypeptide or a fragment thereof in infected hosts (e.g., see Logan & Shenk, Proc. Natl. Acad. Sci. USA, 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA 79:4927-4931, 1982). Of particular interest are vectors based on bovine papilloma virus which have the ability to replicate as extrachromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of a BACE gene in host cells (Cone & Mulligan, Proc. Natl. Acad. Sci. USA, 81:6349-6353, 1984). High level expression may also be achieved using inducible promoters, including, but not limited to, the metallothionine IIA promoter and heat shock promoters.

For long-term, high-yield production of recombinant proteins, stable expression is preferred. Rather than using expression vectors which contain viral origins of replication, host cells can be transformed with the cDNA encoding an APP, APP fragment or BACE polypeptide controlled by appropriate expression control elements (e.g., promoter, enhancer, sequences, transcription terminators, polyadenylation sites, etc.), and a selectable marker. The selectable marker in the recombinant vector confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler, et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes can be employed in tk-, hgprt- or aprt-cells respectively. Also, anti-metabolite resistance can be used as the basis of selection for dhfr, which confers resistance to methotrexate (Wigler, et al., Proc. Natl. Acad. Sci. USA, 77:3567, 1980; O'Hare, et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin, et al., J. Mol. Biol. 150:1, 1981); and hygro, which confers resistance to hygromycin (Santerre, et al., Gene 30:147, 1984) genes. Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA 85:8047, 1988); and ODC (ornithine decarboxylase) which confers resistance to the omithine decarboxylase inhibitor, 2-(difluoromethyl)-DL-ornithine, DFMO (McConlogue L., In: Current Communications in Molecular Biology, Cold Spring Harbor Laboratory, ed., 1987).

The term “primer” as used herein refers to an oligonucleotide, whether natural or synthetic, which is capable of acting as a point of initiation of synthesis when placed under conditions in which primer extension is initiated or possible. Synthesis of a primer extension product which is complementary to a nucleic acid strand is initiated in the presence of nucleoside triphosphates and a polymerase in an appropriate buffer at a suitable temperature. For instance, if a nucleic acid sequence is inferred from a protein sequence, a primer generated to synthesize nucleic acid sequence encoding the protein sequence is actually a collection of primer oligonucleotides containing sequences representing all possible codon variations based on the degeneracy of the genetic code. One or more of the primers in this collection will be homologous with the end of the target sequence. Likewise, if a “conserved” region shows significant levels of polymorphism in a population, mixtures of primers can be prepared that will amplify adjacent sequences.

A polypeptide or protein refers to a polymer in which the monomers are amino acid residues which are joined together through amide bonds. When the amino acids are alpha-amino acids, either the L-optical isomer or the D-optical isomer can be used, the L-isomers being typical. Examples of polypeptides useful in the methods and compositions of the invention include APP (see, for example, Cheler, J. of Neurochemistry 65(4):1431, 1995, which is incorporated herein by reference in its entirety), fragments of APP including Aβ1-40, Aβ1-42, Aβ11-40, and Aβ11-42; and BACE1 (see, e.g., Vassar et al. Science 286:735, 1999, which is incorporated herein by reference in its entirety). Accordingly, the polypeptides of the invention are intended to cover naturally occurring proteins, as well as those which are recombinantly or synthetically synthesized. Polypeptide or protein fragments are also encompassed by the invention. Fragments can have the same or substantially the same amino acid sequence as the naturally occurring protein. A polypeptide or peptide having substantially the same sequence means that an amino acid sequence is largely, but not entirely, the same, but retains a functional activity of the sequence to which it is related. In general polypeptides of the invention include peptides, or full length protein, that contains substitutions, deletions, or insertions into the protein backbone, that would still have an approximately 70%-90% homology to the original protein over the corresponding portion. A yet greater degree of departure from homology is allowed if like-amino acids, i.e. conservative amino acid substitutions, do not count as a change in the sequence

A polypeptide may be substantially related but for a conservative variation, such polypeptides being encompassed by the invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine. The term “conservative variation” also includes the use of a substituted amino acid in place of an unsubstituted parent amino acid provided that antibodies raised to the substituted polypeptide also immunoreact with the unsubstituted polypeptide.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, tissue culture and so on.

Prior to the present invention the role of BACE1 in the processing of APP and fragments thereof were not understood. Accordingly, the invention provides for the first time an understanding of the role of BACE1 in the processing of APP and in AD. Thus, in one embodiment, the invention provides a method for modulating (e.g., inhibiting) the interaction of a BACE1 polypeptide with its substrate APP (either in vitro or in vivo) by administering to a cell or subject an effective amount of a composition which contains a BACE1 polypeptide, or biologically functional fragment thereof an agent (e.g, an antibody, ribozyme, antisense molecule, or double-stranded interfering RNA molecules) that interacts with or inhibits expression or the activity of a BACE1 polypeptide.

As used herein, an “effective amount” of a composition containing a BACE1 polypeptide or a BACE1 polypeptide-modulating agent is defined as that amount that is effective in modulating normal enzymatic activity or interaction of a BACE1 substrate with a BACE1 polypeptide or protein in a subject or cell.

In another embodiment, the present invention provides a method for modulating expression of a BACE1 polypeptide as well as methods for screening for agents which modulate BACE1 polypeptide gene expression. In this embodiment, a cell or subject is contacted with an agent suspected or known to have BACE1 polypeptide expression modulating activity. The change in BACE1 polypeptide gene expression is then measured as compared to a control or standard sample. The control or standard sample can be the baseline expression of the cell or subject prior to contact with the agent. An agent which modulates BACE1 polypeptide gene expression may be a polynucleotide. For example, the polynucleotide may be an antisense, a triplex agent, a ribozyme, or a double-stranded interfering RNA that interacts with a BACE1. For example, an antisense molecule may be directed to the structural gene region or to the promoter region of a BACE1 gene. The agent may be an agonist, antagonist, peptide, peptidomimetic, antibody, or chemical.

Double-stranded interfering RNA molecules are especially useful to inhibit expression of a target gene. For example, double-stranded RNA molecules can be injected into a target cell or organism to inhibit expression of a gene and the resultant gene products activity. It has been found that such double-stranded RNA molecules are more effective at inhibiting expression than either RNA strand alone. (Fire et al., Nature, 1998, 19:391(6669):806-11).

When a disorder is associated with abnormal expression of a BACE1 polypeptide (e.g., overexpression, or expression of a mutated form of the protein) or as a result of expression of a substrate for the BACE1 polypeptide, a therapeutic approach which directly interferes with the translation of a BACE1 polypeptide is possible. Alternatively, similar methodology may be used to study gene activity. For example, antisense nucleic acid, double-stranded interfering RNA or ribozymes could be used to bind to a BACE1 polypeptide mRNA sequence or to cleave it. Antisense RNA or DNA molecules bind specifically with a targeted gene's RNA message, interrupting the expression of that gene's protein product. The antisense binds to the messenger RNA forming a double stranded molecule which cannot be translated by the cell. Antisense oligonucleotides of about 15-25 nucleotides are preferred since they are easily synthesized and have an inhibitory effect just like antisense RNA molecules. In addition, chemically reactive groups, such as iron-linked ethylenediaminetetraacetic acid (EDTA-Fe) can be attached to an antisense oligonucleotide, causing cleavage of the RNA at the site of hybridization. Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target BACE1 polypeptide producing cell. The use of antisense methods to inhibit the in vitro translation of genes is well known in the art (Marcus-Sakura, Anal. Biochem., 172:289, 1988).

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher, et al., Antisense Res. and Dev., 1:227, 1991; Helene, Anticancer Drug Design, 6:569, 1991).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J. Amer. Med. Assn., 260:3030, 1988). A major advantage of this approach is that, because they are sequence-specific, only mRNAs with particular sequences are inactivated.

There are two basic types of ribozymes namely, tetrahymena-type (Hasselhoff, Nature, 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-base recognition sequences are preferable to shorter recognition sequences.

These and other uses of antisense and ribozymes methods to inhibit the in vivo translation of genes are known in the art (e.g., De Mesmaeker, et al., Curr. Opin. Struct. Biol., 5:343, 1995; Gewirtz, A. M., et al., Proc. Natl. Acad. Sci. U.S.A., 93:3161, 1996b; Stein, C. A., Chem. and Biol. 3:319, 1996).

Delivery of antisense, triplex agents, ribozymes, competitive inhibitors, double-stranded interfering RNA and the like can be achieved using a recombinant expression vector such as a chimeric virus or a colloidal dispersion system or by injection. Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a polynucleotide sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing, for example, an antisense polynucleotide.

Another targeted delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes has been classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. In general, the compounds bound to the surface of the targeted delivery system will be ligands and receptors which will allow the targeted delivery system to find and “home in” on the desired cells. A ligand may be any compound of interest which will bind to another compound, such as a receptor.

The agents useful in the method of the invention can be administered, for in vivo application, parenterally by injection or by gradual perfusion over time. Administration may be intravenously, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents and inert gases and the like.

It is envisioned that the invention can be used to treat pathologies associated with neurodegenerative diseases and associated disorders, Aβ11-40/42 accumulation diseases (e.g., Alzheimer's Disease). Therefore, the present invention encompasses methods for ameliorating a disorder associated with neurodegenerative disorders, including treating a subject having the disorder, at the site of the disorder, with an agent which modulates a BACE1 expression or activity or its interaction with its substrate (e.g., APP). Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure for an infection or disease and/or adverse effect attributable to the infection or disease. “Treating” as used herein covers any treatment of, or prevention of a disease in an invertebrate, a vertebrate, a mammal, particularly a human, and includes: (a) preventing the disorder from occurring in a subject that may be predisposed to the disorder, but has not yet been diagnosed as having it; (b) inhibiting the disorder, i.e., arresting its development; or (c) relieving or ameliorating the disorder, i.e., cause regression of the disorder. By “Aβ11-40/42 accumulation disease” is meant a disease that is characterized as having an increase in Aβ11-40 and Aβ11-42 peptides over normal levels. Such accumulations in APP fragments lead to degenerative diseases that include, for example, Alzheimer's Disease.

The invention includes various pharmaceutical compositions useful for ameliorating symptoms attributable to a BACE1 or APP processing associated disorder. The pharmaceutical compositions according to one embodiment of the invention are prepared by bringing an antibody against a BACE1 polypeptide, a polypeptide or peptide derivative of a BACE1 polypeptide, a BACE1 polypeptide mimetic, a drug, chemical or combination of chemicals or a BACE1 polypeptide-modulating agent into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dose units. Solid dose units are tablets, capsules and suppositories. For treatment of a subject, depending on activity of the compound, manner of administration, nature and severity of the disorder, age and body weight of the subject, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249:1527, (1990); Gilman et al. (eds.) (1990), each of which is herein incorporated by reference.

“Administering” the pharmaceutical composition of the present invention may be accomplished by any means known to the skilled artisan. Preferably a “subject” refers to a mammal, most preferably a human, but may be any organism.

An anti-BACE1 antibody can be administered parenterally, enterically, by injection, rapid infusion, nasopharyngeal absorption, dermal absorption, rectally and orally. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water.

In another embodiment, the invention provides a method for identifying an agent which interacts with or modulates expression or activity of a BACE1 polypeptide including incubating components comprising an agent and a BACE1 polypeptide, or a recombinant cell expressing a BACE1 polypeptide, under conditions sufficient to allow the agent to interact and determining the affect of the agent on the expression or activity of the gene or polypeptide, respectively. The term “affect”, as used herein, encompasses any means by which gene expression or protein activity can be modulated, and includes measuring the interaction of the agent with the BACE1 protein by physical means including, for example, fluorescence detection of the binding of a the protein to a substrate or binding agent. Such agents can include, for example, polypeptides, peptidomimetics, chemical compounds, small molecules and biologic agents as described below.

Incubating includes conditions which allow contact between the test agent and a BACE1 polypeptide, a cell expressing a BACE1 polypeptide or nucleic acid encoding a BACE1 polypeptide. Contacting includes in solution and in solid phase. The test agent may optionally be a combinatorial library for screening a plurality of agents. Agents identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), oligonucleotide ligation assays (OLAs) (Landegren, et al., Science, 241:1077, 1988), and the like. Molecular techniques for DNA analysis have been reviewed (Landegren, et al., Science, 242:229-237, 1988). Thus, the methods of the invention includes combinatorial chemistry methods for identifying chemical agents that bind to or affect BACE1 polypeptide expression or activity.

Areas of investigation are the development of therapeutic treatments. The screening identifies agents that provide modulation of BACE1 polypeptide function in targeted organisms. Of particular interest are screening assays for agents that have a low toxicity or a reduced number of side effects for humans. In particular, since the invention provides for the first time that BACE1 activity is species specific and results in the formation of an Aβ11-40/42 product, detection of the effect of an agent on product formation can be easily assayed and thus the identification of potential therapeutics is provided by the present invention.

The term “agent” as used herein describes any molecule, e.g. protein or pharmaceutical, with the capability of altering or mimicking the physiological function or expression of a BACE1 polypeptide. Generally, a plurality of assay mixtures are run in parallel with different agent concentrations to obtain a differential response to the various concentrations. Typically, one of these concentrations serves as a negative control, i.e. at zero concentration or below the level of detection.

In a further embodiment, the invention provides a method of detecting a BACE1 or APP fragments (e.g., Aβ11-40/42), a BACE1 or APP (e.g., Aβ11-40/42) polypeptide or a BACE1 polynucleotide or diagnosing a BACE1 or APP fragments (e.g., Aβ11-40/42)-related disorder (e.g., AD) in a subject including contacting a sample (e.g., blood, serum, cerebrospinal fluid or a cellular sample, or tissue sample) suspected of containing a BACE1 or APP (e.g., Aβ11-40/42) polypeptide or a BACE1 polynucleotide with a reagent which binds to the polypeptide or polynucleotide (herein after sample). The sample can be or contain a nucleic acid, such as DNA or RNA, or a protein. When the sample contains a nucleic acid, the reagent is a nucleic acid probe or PCR primer. When the sample contains protein, the reagent is an antibody probe. The probes are detectably labeled, for example, with a radioisotope, a fluorescent compound, a bioluminescent compound, a chemiluminescent compound, a metal chelator or an enzyme. Those of ordinary skill in the art will know of other labels suitable for binding to an antibody or nucleic acid probe, or will be able to ascertain such, using routine experimentation. There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds. In addition, the antibodies, polypeptides and polynucleotide sequences of the invention can be used to diagnosis a BACE1 or APP (e.g., Aβ11-40/42)-related disorder.

A monoclonal antibody of the invention, directed toward a BACE1 or APP (e.g., Aβ11-40/42) polypeptide is useful for the in vivo and in vitro detection of antigen. The detectably labeled monoclonal antibody is given in a dose, which is diagnostically effective. The term “diagnostically effective” means that the amount of detectably labeled monoclonal antibody is administered in sufficient quantity to enable detection of a BACE1 or APP fragments (e.g., Aβ11-40/42) or a BACE1 or APP (e.g., Aβ11-40/42) polypeptide antigen for which the monoclonal antibodies are specific.

The concentration of a detectably labeled monoclonal antibody administered to a subject should be sufficient such that the binding to those cells, body fluid, or tissue having a BACE1 or APP (e.g., Aβ11-40/42) polypeptide that is detectable compared to the background. Further, it is desirable that the detectably labeled monoclonal antibody be rapidly cleared from the circulatory system in order to give the best target-to-background signal ratio.

For in vivo diagnostic imaging, the type of detection instrument available is a major factor in selecting a given radioisotope. The radioisotope chosen must have a type of decay, which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 key range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr, and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements, which are particularly useful in such techniques, include ¹⁵⁷Gd, ⁵⁵Mn, ¹⁶²Dy, ⁵²Cr, and ⁵⁶Fe.

In another embodiment, nucleic acid probes can be used to identify a BACE1 polynucleotide from a sample obtained from a subject. Examples of specimens from which nucleic acid sequence encoding a BACE1 polypeptide can be derived include insect, human, primate, swine, porcine, feline, canine, equine, murine, cervine, caprine, lupine, leporidine, opine and bovine species.

In another embodiment, nucleic acid probes can be used to identify a polynucleotide encoding a BACE1 polypeptide from a specimen obtained from a subject. Examples of specimens from which nucleic acid sequence encoding a BACE1 polypeptide can be derived include human, primate, swine, porcine, feline, canine, equine, murine, cervine, caprine, lupine, leporidine and bovine species.

Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res. 9:879, 1981).

In an embodiment of the invention, purified nucleic acid fragments containing intervening sequences or oligonucleotide sequences of 10-50 base pairs are radioactively labeled. The labeled preparations are used to probe nucleic acids from a specimen by the Southern hybridization technique. Nucleotide fragments from a specimen, before or after amplification, are separated into fragments of different molecular masses by gel electrophoresis and transferred to filters that bind nucleic acid. After exposure to the labeled probe, which will hybridize to nucleotide fragments containing target nucleic acid sequences, binding of the radioactive probe to target nucleic acid fragments is identified by autoradiography (see Genetic Engineering, 1, ed. Robert Williamson, Academic Press, (1981), 72-81). Alternatively, nucleic acid from the specimen can be bound directly to filters to which the radioactive probe selectively attaches by binding nucleic acids having the sequence of interest. Specific sequences and the degree of binding is quantitated by directly counting the radioactive emissions.

Where the target nucleic acid is not amplified, detection using an appropriate hybridization probe may be performed directly on the separated nucleic acid. In those instances where the target nucleic acid is amplified, detection with the appropriate hybridization probe would be performed after amplification.

For the most part, the probe will be detectably labeled with an atom or inorganic radical, most commonly using radionuclides, but also heavy metals can be used. Conveniently, a radioactive label may be employed. Radioactive labels include ³²P, ¹²⁵I, ³H, ¹⁴C, ¹¹¹In, ⁹⁹Tc, or the like. Any radioactive label may be employed which provides for an adequate signal and has sufficient half-life. Other labels include ligands, which can serve as a specific binding pair member for a labeled ligand, and the like. A wide variety of labels routinely employed in immunoassays can readily be employed in the present assay. The choice of the label will be governed by the effect of the label on the rate of hybridization and binding of the probe to a nucleotide sequence. It will be necessary that the label provide sufficient sensitivity to detect the amount of a nucleotide sequence available for hybridization.

The manner in which the label is bound to the probe will vary depending upon the nature of the label. For a radioactive label, a wide variety of techniques can be employed. Commonly employed is nick translation with an a ³²P-dNTP or terminal phosphate hydrolysis with alkaline phosphatase followed by labeling with radioactive ³²P employing ³²P-NTP and T4 polynucleotide kinase. Alternatively, nucleotides can be synthesized where one or more of the elements present are replaced with a radioactive isotope, e.g., hydrogen with tritium. If desired, complementary labeled strands can be used as probes to enhance the concentration of hybridized label.

Standard hybridization techniques for detecting a nucleic acid sequence are known in the art. The particular hybridization technique is not essential to the invention. Other hybridization techniques are described by Gall and Pardue, Proc. Natl. Acad. Sci. 63:378, 1969); and John, et al., Nature, 223:582, 1969). As improvements are made in hybridization techniques they can readily be applied in the method of the invention.

The amount of labeled probe present in the hybridization solution will vary widely, depending upon the nature of the label, the amount of the labeled probe that can reasonably bind to the filter, and the stringency of the hybridization. Generally, substantial excess over stoichiometric concentrations of the probe will be employed to enhance the rate of binding of the probe to the fixed target nucleic acid.

The materials for use in the assay of the invention are ideally suited for the preparation of a kit. Such a kit may comprise a carrier means containing one or more container means such as vials, tubes, and the like, each of the container means comprising one of the separate elements to be used in the method. One of the container means may comprise a probe which is or can be detectably labeled. Such probe may be a nucleic acid sequence specific for BACE1; or antibodies specific for BACE1, fragments thereof, or APP or fragments thereof.

The kit may also contain a container comprising a reporter-means, such as an enzymatic, fluorescent, or radionucleotide label to identify the detectably labeled oligonucleotide probe or antibody.

Where the kit utilizes nucleic acid hybridization to detect the target nucleic acid, the kit may also have containers containing nucleotide(s) for amplification of the target nucleic acid sequence.

Various methods to make the transgenic non-human animals of the invention can be employed. Generally speaking, three such methods may be employed. In one such method, an embryo at the pronuclear stage (a “one cell embryo”) is harvested from a female and the transgene is microinjected into the embryo, in which case the transgene will be chromosomally integrated into both the germ cells and somatic cells of the resulting mature animal. In another such method, embryonic stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. In yet another such method, embryonic cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Therefore, of the methods to make transgenic animals described generally above, retrovirus infection is preferred for avian species, for example as described in U.S. Pat. No. 5,162,215. If micro-injection is to be used with avian species, however, a published procedure by Love et al., (Biotechnology, 12, January 1994) can be utilized whereby the embryo is obtained from a sacrificed hen approximately two and one-half hours after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization.

The “non-human animals” of the invention include bovine, porcine, ovine and avian animals (e.g., cow, pig, sheep, chicken). The “transgenic non-human animals” of the invention are produced by introducing “transgenes” into the germline of the non-human animal. Embryonal target cells at various developmental stages can be used to introduce transgenes. Different methods are used depending on the stage of development of the embryonal target cell. The zygote is the best target for micro-injection. The use of zygotes as is target for gene transfer has a major advantage in that in most cases the injected DNA will be incorporated into the host gene before the first cleavage (Brinster et al., Proc. Natl. Acad. Sci. USA 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

The term “transgenic” is used to describe an animal which includes exogenous genetic material within all of its cells. A “transgenic” animal can be produced by cross-breeding two chimeric animals which include exogenous genetic material within cells used in reproduction. Twenty-five percent of the resulting offspring will be transgenic i.e., animals which include the exogenous genetic material within all of their cells in both alleles, 50% of the resulting animals will include the exogenous genetic material within one allele and 25% will include no exogenous genetic material.

In the microinjection method useful in the practice of the subject invention, the transgene is digested and purified free from any vector DNA, e.g., by gel electrophoresis. It is preferred that the transgene include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionin, skeletal actin, P-enolpyruvate carboxylase (PEPCK), phosphoglycerate (PGK), DHFR, and thymidine kinase. Promoters for viral long terminal repeats (LTRs) such as Rous Sarcoma Virus can also be employed. When the animals to be made transgenic are avian, preferred promoters include those for the chicken β-globin gene, chicken lysozyme gene, and avian leukosis virus. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce transgene into a non-human animal, as described above. The developing non-human embryo can be cultured in vitro to the blastocyst stage. During this time, the blastomeres can be targets for retroviral infection (Jaenich, R., Proc. Natl. Acad. Sci USA 73:1260-1264, 1976). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retro virus carrying the transgene (Jahner, et al., Proc. Natl. Acad. Sci. USA 82: 6927-6931, 1985; Van der Putten, et al., Proc. Natl. Acad. Sci USA 82: 6148-6152, 1985). Transfection is easily and efficiently obtained by culturing the blastomeres on a monolayer of virus-producing cells (Van der Putten, supra; Stewart, et al., EMBO J. 6: 383-388, 1987). Alternatively, infection can be performed at a later stage. Virus or virus-producing cells can be injected into the blastocoele (D. Jahner et al., Nature 298: 623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

A third type of target cell for transgene introduction is the embryonal stem cell (ES). ES cells are obtained from pre-implantation embryos cultured in vitro and fused with embryos (M. J. Evans et al., Nature 292:154-156, 1981; M. O. Bradley et al., Nature 309:255-258, 1984; Gossler, et al., Proc. Natl. Acad. Sci USA 83:9065-9069, 1986; and Robertson et al., Nature 322:445-448, 1986). Transgenes can be efficiently introduced into the ES cells by DNA transfection or by retro virus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, R., Science 240:1468-1474, 1988).

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

“Transgene” means any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences which encode BACE1 or a selectable marker flanked by regions of sequence having homology to BACE1, and include polynucleotides, which may be expressed in a transgenic non-human animal. The term “transgenic” as used herein additionally includes any organism whose genome has been altered by in vitro manipulation of the early embryo or fertilized egg or by any transgenic technology to induce a specific gene knockout. The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination. As used herein, the term “transgenic” includes any transgenic technology familiar to those in the art which can produce an organism carrying an introduced transgene or one in which an endogenous gene has been rendered non-functional or “knocked out.”

After an embryo has been microinjected, colonized with transfected embryonic stem cells or infected with a retrovirus containing the transgene (except for practice of the subject invention in avian species which is addressed elsewhere herein) the embryo is implanted into the oviduct of a pseudopregnant female. The consequent progeny are tested for incorporation of the transgene by Southern blot analysis of blood or tissue samples using transgene specific probes. PCR is particularly useful in this regard. Positive progeny (GO) are crossbred to produce offspring (GI) which are analyzed for transgene expression by Northern blot analysis of tissue samples.

The animals contemplated for use in the practice of the subject invention include, rattus sp., avian sp. canine sp., non-human primate sp., feline sp., mouse sp. etc. For purposes of the subject invention, these animals are referred to as “transgenic” when such animal has had a heterologous DNA sequence, or one or more additional DNA sequences normally endogenous to the animal (collectively referred to herein as “transgenes”) chromosomally integrated into the germ cells of the animal. The transgenic animal (including its progeny) will also have the transgene fortuitously integrated into the chromosomes of somatic cells.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, a “heterologous gene” or “heterologous polynucleotide sequence” is defined in relation to the transgenic non-human organism producing or containing such a gene product. A heterologous polypeptide is defined as a polypeptide having an amino acid sequence or an encoding DNA sequence corresponding to that of a heterologous gene not normally found in an organism.

As used herein, the term “targeting construct” refers to a polynucleotide which comprises: (1) at least one homology region having a sequence that is substantially identical to or substantially complementary to a sequence present in a host cell endogenous gene locus, and (2) a targeting region which becomes integrated into an host cell endogenous gene locus by homologous recombination between a targeting construct homology region and the endogenous gene locus sequence. A transiently incorporated targeting construct is one that is incorporated into the endogenous gene locus and is eliminated from the host genome by selection. A targeting region may comprise a sequence that is substantially homologous to an endogenous gene sequence and/or may comprise a non-homologous sequence, such as a selectable marker (e.g., neo, tk, gpt). The term “targeting construct” does not necessarily indicate that the polynucleotide comprises a gene which becomes integrated into the host genome, nor does it necessarily indicate that the polynucleotide comprises a complete structural gene sequence. As used in the art, the term “targeting construct” is synonymous with the term “targeting transgene” as used herein.

The term “homology region” as used herein refer to a segment (i.e., a portion) of a targeting construct having a sequence that substantially corresponds to, or is substantially complementary to, a predetermined endogenous gene sequence, which can include sequences flanking said gene. A homology region is generally at least about 100 nucleotides long, preferably at least about 250 to 500 nucleotides long, typically at least about 1000 nucleotides long or longer. Although there is no demonstrated theoretical minimum length for a homology region to mediate homologous recombination, it is believed that homologous recombination efficiency generally increases with the length of the homology region. Similarly, the recombination efficiency increases with the degree of sequence homology between a targeting construct homology region and the endogenous target sequence, with optimal recombination efficiency occurring when a homology region is isogenic with the endogenous target sequence. A homology region does not necessarily denote formation of a base-paired hybrid structure with an endogenous sequence. Endogenous gene sequences that substantially correspond to, or are substantially complementary to, a transgene homology region are referred to herein as “crossover target sequences” or “endogenous target sequences.”

As used herein, the term “transcriptional unit” or “transcriptional complex” refers to a polynucleotide sequence that comprises a structural gene (exons), a cis-acting linked regulatory element (e.g., a promoter or enhancer sequence) and other cis-acting sequences necessary for efficient transcription of the structural sequences, distal regulatory elements necessary for appropriate tissue-specific and developmental transcription of the structural sequences, and additional cis sequences important for efficient transcription and translation (e.g., polyadenylation site, mRNA stability controlling sequences).

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

A correctly targeted construct integrates within or adjacent to an endogenous crossover target sequence, such as a portion of an endogenous BACE1 gene locus. For example, a targeting transgene encoding neo and which is flanked by homology regions having substantial identity with endogenous BACE1 gene sequences of the first exon of BACE1 is correctly targeted when the transgene portion is integrated into a chromosomal location so as to replace, for example, the first exon of the endogenous BACE1 gene. It is possible to generate cells having both a correctly targeted transgene(s) and an incorrectly targeted transgene(s). Cells and animals having a correctly targeted transgene(s) and/or an incorrectly targeted transgene(s) may be identified and resolved by PCR and/or Southern blot analysis of genomic DNA.

As used herein, the term “targeting region” refers to a portion of a targeting construct which becomes integrated into an endogenous chromosomal location following homologous recombination between a homology region and an endogenous BACE1 gene sequence. Typically, a targeting region is flanked on each side by a homology region, such that a double-crossover recombination between each of the homology regions and their corresponding endogenous BACE1 gene sequences results in replacement of the portion of the endogenous BACE1 gene locus by the targeting region; in such double-crossover gene replacement targeting constructs the targeting region can be referred to as a “replacement region”. However, some targeting constructs may employ only a single homology region.

As used herein, the term “replacement region” refers to a portion of a targeting construct flanked by homology regions. Upon double-crossover homologous recombination between flanking homology regions and their corresponding endogenous BACE1 gene crossover target sequences, the replacement region is integrated into the host cell chromosome between the endogenous crossover target sequences. Replacement regions can be homologous (e.g., have a sequence similar to the endogenous BACE1 gene sequence but having a point mutation or missense mutation), non-homologous (e.g., a neo gene expression cassette), or a combination of homologous and non-homologous regions. The replacement region can convert the endogenous BACE1 allele into an mutant BACE1 allele comprising a point mutation or missense mutation or disrupt the BACE1 allele by integrating a non-homologous transgene at the BACE1 allele.

The terms “functional disruption” or “functionally disrupted” as used herein means that a gene locus comprises at least one mutation or structural alteration such that the functionally disrupted gene is incapable of directing the efficient expression of functional gene product. For example, an endogenous BACE1 gene that has a neo gene cassette integrated into an exon of a BACE1 gene, is not capable of encoding a functional protein and is therefore a functionally disrupted BACE1 gene locus. In addition, a targeted mutation in an exon of an endogenous BACE1 gene may result in a mutated endogenous gene that can express a truncated BACE1 protein that is non-functional. Functional disruption can include the complete substitution of a heterologous BACE1 gene locus in place of an endogenous BACE1 locus, so that, for example, a targeting transgene that replaces the entire mouse BACE1 locus with a human BACE1 allele, which may be functional in the mouse, is said to have functionally disrupted the endogenous murine BACE1 locus by displacing it. Preferably, at least one exon which is incorporated into the mRNAs encoding most or all of the BACE1 isoforms are functionally disrupted. Deletion or interruption of essential transcriptional regulatory elements, polyadenylation signal(s), splicing site sequences will also yield a functionally disrupted gene. Functional disruption of an endogenous BACE1 gene, may also be produced by other methods (e.g., antisense polynucleotide gene suppression). The term “structurally disrupted” refers to a targeted gene wherein at least one structural sequence (e.g., an exon sequence) has been altered by homologous gene targeting (e.g., by insertion, deletion, point mutation(s), and/or rearrangement). Typically, BACE1 alleles that are structurally disrupted are consequently functionally disrupted, however BACE1 alleles may also be functionally disrupted without concomitantly being structurally disrupted, i.e., by targeted alteration of a non-exon sequence such as ablation of a promoter. An allele comprising a targeted alteration that interferes with the efficient expression of a functional gene product from the allele is referred to in the art as a “null allele” or “knockout allele”.

The term “agent” is used herein to denote a chemical compound, a mixture of chemical compounds, a biological macromolecule (e.g., a peptide, peptidomimetic, or antibody), or an extract made from biological materials such as bacteria, plants, fungi, or animal (particularly mammalian) cells or tissues.

As used herein, “isoform”, “BACE1”, and “BACE1 isoform” refer to a polypeptide that is encoded by at least one exon and includes a sequence as set forth in GenBank Accession No. AF190725 (Vassar et al., Science 286:735, 1999). A BACE isoform may be encoded by an BACE allele (or exon thereof) that is associated with a form of Alzheimer's disease or that is not associated with an AD disease phenotype.

In some embodiments, the endogenous non-human BACE1 alleles are functionally disrupted so that expression of endogenously encoded BACE1 is suppressed or eliminated. In one variation, an endogenous BACE1 allele is targeted for disruption by homologous recombination.

Gene targeting, which is a method of using homologous recombination to modify a mammalian genome, can be used to introduce changes into cultured cells. By targeting a gene of interest in embryonic stem (ES) cells, these changes can be introduced into the germlines of laboratory animals to study the effects of the modifications on whole organisms, among other uses. The gene targeting procedure is accomplished by introducing into tissue culture cells a DNA targeting construct that has a segment homologous to a target locus and which also comprises an intended sequence modification (e.g., insertion, deletion, point mutation). The treated cells are then screened for accurate targeting to identify and isolate those which have been properly targeted. A common scheme to disrupt gene function by gene targeting in ES cells is to construct a targeting construct which is designed to undergo a homologous recombination with its chromosomal counterpart in the ES cell genome. The targeting constructs are typically arranged so that they insert additional sequences, such as a selectable marker, into coding elements of the target gene, thereby functionally disrupting it. Targeting constructs usually are insertion-type or replacement-type constructs (Hasty et al., Mol. Cell. Biol. 11:4509, 1991).

The invention encompasses methods to produce non-human animals (e.g., non-primate mammals) that have the endogenous BACE1 gene inactivated by gene targeting with a homologous recombination targeting construct. Typically, a non-human BACE1 gene sequence is used as a basis for producing PCR primers that flank a region that will be used as a homology region in a targeting construct. The PCR primers are then used to amplify, by high fidelity PCR amplification (Mattila et al., Nucleic Acids Res. 19:4967, 1991; Eckert, K. A. and Kunkel, T. A., PCR Methods and Applications 1:17, 1991; U.S. Pat. No. 4,683,202, which are incorporated herein by reference), a genomic sequence from a genomic clone library or from a preparation of genomic DNA, preferably from the strain of non-human animal that is to be targeted with the targeting construct. The amplified DNA is then used as a homology region and/or targeting region. Thus, homology regions for targeting a non-human BACE1 gene may be readily produced on the basis of nucleotide sequence information available in the art and/or by routine cloning (e.g., GenBank Accession No. AF190725). General principles regarding the construction of targeting constructs and selection methods are reviewed in Bradley et al., Bio/Technology 10:534, 1992, incorporated herein by reference).

In addition, to the disruption of endogenous non-human BACE1 genes the transgenic organism may include one or more transgenes encoding for example APP comprising the Swedish mutation.

Targeting constructs can be transferred into pluripotent stem cells, such as murine embryonal stem cells, wherein the targeting constructs homologously recombine with a portion of an endogenous BACE1 gene locus and create mutation(s) (i.e., insertions, deletions, rearrangements, sequence replacements, and/or point mutations) which prevent the functional expression of the endogenous BACE1 gene.

A preferred method of the invention is to delete, by targeted homologous recombination, essential structural elements of the endogenous BACE1 gene. For example, a targeting construct can homologously recombine with an endogenous BACE1 gene and delete a portion spanning substantially all of one or more of the exons to create an exon-depleted allele, typically by inserting a replacement region lacking the corresponding exon(s). Transgenic animals homozygous for the exon-depleted allele (e.g., by breeding of heterozygotes to each other) produce cells which are essentially incapable of expressing a functional endogenous BACE1 polypeptide (preferably incapable of expressing any of the naturally-occurring isoforms). Similarly, homologous gene targeting can be used, if desired, to functionally disrupt a BACE1 gene by deleting only a portion of an exon.

Targeting constructs can also be used to delete essential regulatory elements of an endogenous BACE1 gene, such as promoters, enhancers, splice sites, polyadenylation sites, and other regulatory sequences, including cis-acting sequences that occur upstream or downstream of the BACE1 structural gene but which participate in endogenous BACE1 gene expression. Deletion of regulatory elements is typically accomplished by inserting, by homologous double-crossover recombination, a replacement region lacking the corresponding regulatory element(s).

Another method of the invention is to interrupt essential structural and/or regulatory elements of an endogenous BACE1 gene by targeted insertion of a polynucleotide sequence, and thereby functionally disrupt the endogenous BCE1 gene. For example, a targeting construct can homologously recombine with an endogenous BACE1 gene and insert a non-homologous sequence, such as a neo expression cassette, into a structural element (e.g., an exon) and/or regulatory element (e.g., enhancer, promoter, splice site, polyadenylation site) to yield a targeted BCE1 allele having an insertional interruption. The inserted sequence can range in size from about 1 nucleotide (e.g., to produce a frameshift in an exon sequence) to several kilobases or more, as limited by efficiency of homologous gene targeting with targeting constructs having a long nonhomologous replacement region.

Targeting constructs of the invention can also be employed to replace a portion of an endogenous BACE1 gene with an exogenous sequence (i.e., a portion of a targeting transgene); for example, an exon of a BACE1 gene may be replaced with a substantially identical portion that contains a nonsense or missense mutation.

In one embodiment, inactivation of an endogenous murine BACE1 locus is achieved by targeted disruption of the appropriate gene by homologous recombination in a mouse embryonic stem cell. For inactivation, any targeting construct that produces a genetic alteration in the target BACE1 gene locus resulting in the prevention of effective expression of a functional gene product of that locus may be employed. If only regulatory elements are targeted, some low-level expression of the targeted gene may occur (i.e., the targeted allele is “leaky”), however the level of expression may be sufficiently low that the leaky targeted allele is functionally disrupted.

In one embodiment of the invention, an endogenous BACE1 gene in a non-human host is functionally disrupted by homologous recombination with a targeting construct that does not comprise a functionally equivalent sequence. In this embodiment, a portion of the targeting construct integrates into an essential structural or regulatory element of the endogenous BACE1 gene locus, thereby functionally disrupting it to generate a null allele. Typically, null alleles are produced by integrating a non-homologous sequence encoding a selectable marker (e.g., a neo gene expression cassette) into an essential structural and/or regulatory sequence of a BACE1 gene by homologous recombination of the targeting construct homology regions with endogenous BACE1 gene sequences, although other strategies may be employed.

Most usually, a targeting construct is transferred by electroporation or microinjection into a totipotent embryonal stem (ES) cell line, such as the murine AB-1 or CCE lines. The targeting construct homologously recombines with endogenous sequences in or flanking an BACE1 gene locus and functionally disrupts at least one allele of the BACE1 gene. Typically, homologous recombination of the targeting construct with endogenous BACE1 locus sequences results in integration of a non-homologous sequence encoding a selectable marker, such as neo, usually in the form of a positive selection cassette. The functionally disrupted allele is termed an BACE1 null allele. ES cells having at least one BACE1 null allele are selected for by propagating the cells in a medium that permits the preferential propagation of cells expressing the selectable marker. Selected ES cells are examined by PCR analysis and/or Southern blot analysis to verify the presence of a correctly targeted BACE1 allele. Breeding of non-human animals which are heterozygous for a null allele may be performed to produce non-human animals homozygous for said null allele, so-called “knockout” animals (Donehower et al., Nature 256:215, 1992; incorporated herein by reference). In some instances, breeding animals to maintain heterozygosity may be desired. As described more fully below, the transgenic organisms of the invention have utility as both heterozygous and homozygous BACE1 null alleles. Alternatively, ES cells homozygous for a null allele having an integrated selectable marker can be produced in culture by selection in a medium containing high levels of the selection agent (e.g., G418 or hygromycin). Heterozygosity and/or homozygosity for a correctly targeted null allele can be verified with PCR analysis and/or Southern blot analysis of DNA isolated from an aliquot of a selected ES cell clone and/or from tail biopsies.

If desired, a transgene encoding, for example, a heterologous APP polypeptide comprising the Swedish mutation can be transferred into a non-human host having a BACE1 null allele, preferably into a non-human ES cell that is homozygous for the BACE1 null allele. It is generally advantageous that the transgene comprises a promoter and enhancer which drive expression of structural sequences encoding a functional heterologous Swedish mutation APP gene product. Thus, for example, a knockout mouse homozygous for null alleles at the BACE1 locus can serve as a host for a transgene which encodes and expresses a gene associated with an Alzheimer's Disease Associated phenotype.

Several gene targeting techniques have been described, including but not limited to: co-electroporation, single-crossover integration, and double-crossover recombination (Bradley et al., Bio/Technology 10:534, 1992). The invention can be practiced using essentially any applicable homologous gene targeting strategy known in the art. The configuration of a targeting construct depends upon the specific targeting technique chosen. For example, a targeting construct for single-crossover integration targeting need only have a single homology region linked to the targeting region, whereas a double-crossover replacement-type targeting construct requires two homology regions, one flanking each side of the replacement region.

For example, in one embodiment a targeting construct comprising, in order: (1) a first homology region having a sequence substantially identical to a sequence within about 3 kilobases upstream (i.e., in the direction opposite to the translational reading frame of the exons) of an exon of an endogenous BACE1 gene, (2) a replacement region comprising a positive selectable marker (e.g., a pgk promoter driving transcription of a neo gene), (3) a second homology region having a sequence substantially identical to a sequence within about 2 kilobases downstream of said exon of said endogenous BACE1 gene, and (4) a negative selectable marker (e.g., a HSV tk promoter driving transcription of an HSV tk gene). Such a targeting construct is suitable for double-crossover replacement recombination which deletes a portion of the endogenous BACE1 locus spanning the desired exon and replaces it with the replacement region having the positive selectable marker. If the deleted exon is essential for expression of a functional BACE1 gene product, the resultant exon-depleted allele is functionally disrupted and is termed a null allele.

Targeting constructs of the invention comprise at least one BACE1 homology region operably linked to a targeting region. A homology region has a sequence which substantially corresponds to, or is substantially complementary to, an endogenous BACE1 gene sequence of a non-human host animal, and may comprise sequences flanking the BACE1 gene.

Although no lower or upper size boundaries for recombinant homology regions for gene targeting have been identified in the art, the typical homology region is believed to be in the range between about 50 base pairs and several tens of kilobases. Thus, targeting constructs are generally at least about 50 to 100 nucleotides long, preferably at least about 250 to 500 nucleotides long, more preferably at least about 1000 to 2000 nucleotides long, or longer. Construct homology regions are generally at least about 50 to 100 bases long, preferably at least about 100 to 500 bases long, and more preferably at least about 750 to 2000 bases long. It is believed that homology regions of about 7 to 8 kilobases in length are preferred, with one preferred embodiment having a first homology region of about 7 kilobases flanking one side of a replacement region and a second homology region of about 1 kilobase flanking the other side of said replacement region. The length of homology (e.g., substantial identity) for a homology region may be selected at the discretion of the practitioner on the basis of the sequence composition and complexity of the endogenous BACE1 gene target sequence(s) and guidance provided in the art. Targeting constructs have at least one homology region having a sequence that substantially corresponds to, or is substantially complementary to, an endogenous BACE1 gene sequence (e.g., an exon sequence, an enhancer, a promoter, an intronic sequence, or a flanking sequence within about 3-20 kb of a BACE1 gene or BACE1 gene homologue). Such a targeting transgene homology region serves as a template for homologous pairing and recombination with substantially identical endogenous BACE1 gene sequence(s). In targeting constructs, such homology regions typically flank the replacement region, which is a region of the targeting construct that is to undergo replacement with the targeted endogenous BACE1 gene sequence. Thus, a segment of the targeting construct flanked by homology regions can replace a segment of an endogenous BACE 1 gene sequence by double-crossover homologous recombination. Homology regions and targeting regions are linked together in conventional linear polynucleotide linkage (5′ to 3′ phosphodiester backbone). Targeting constructs are generally double-stranded DNA molecules, most usually linear.

Without wishing to be bound by any particular theory of homologous recombination or gene conversion, it is believed that in such a double-crossover replacement recombination, a first homologous recombination (e.g., strand exchange, strand pairing, strand scission, strand ligation) between a first targeting construct homology region and a first endogenous BACE1 gene sequence is accompanied by a second homologous recombination between a second targeting construct homology region and a second endogenous BACE1 gene sequence, thereby resulting in the portion of the targeting construct that was located between the two homology regions replacing the portion of the endogenous BACE1 that was located between the first and second endogenous BACE1 sequences. For this reason, homology regions are generally used in the same orientation (i.e., the upstream direction is the same for each homology region of a transgene to avoid rearrangements). Double-crossover replacement recombination thus can be used to delete a portion of an endogenous BACE1 gene and concomitantly transfer a non-homologous portion (e.g., a neo gene expression cassette) into the corresponding chromosomal location. Double-crossover recombination can also be used to add a non-homologous portion into an endogenous BACE1 gene without deleting endogenous chromosomal portions. However, double-crossover recombination can also be employed simply to delete a portion of an endogenous BACE1 gene sequence without transferring a non-homologous portion into the endogenous BACE1 gene. Upstream and/or downstream from the nonhomologous portion may be a gene which provides for identification of whether a double-crossover homologous recombination has occurred; such a gene is typically the HSV tk gene which may be used for negative selection.

The positive selectable marker encodes a selectable marker which affords a means for selecting cells which have integrated targeting transgene sequences. The negative selectable marker encodes a selectable marker which affords a means for selecting cells which do not have an integrated copy of the negative selection expression cassette. Thus, by a combination positive-negative selection protocol, it is possible to select cells that have undergone homologous replacement recombination and incorporated the portion of the transgene between the homology regions (i.e., the replacement region) into a chromosomal location by selecting for the presence of the positive marker and for the absence of the negative marker. Preferred selectable markers for inclusion in the targeting constructs of the invention encode and express a selectable drug resistance marker and/or a HSV thymidine kinase enzyme. Suitable drug resistance genes include, for example: gpt (xanthine-guanine phosphoribosyltransferase), which can be selected for with mycophenolic acid; neo (neomycin phosphotransferase), which can be selected for with G418 or hygromycin; and DFHR (dihydrofolate reductase), which can be selected for with methotrexate (Mulligan and Berg (1981) Proc. Matl. Acad. Sci. (U.S.A.) 78: 2072; southern and Berg (1982) J. Mol. Appl. Genet. 1: 327; which are incorporated herein by reference).

Selection for correctly targeted recombinants will generally employ at least positive selection, wherein a non-homologous expression cassette encodes and expresses a functional protein (e.g., neo or gpt) that confers a selectable phenotype to targeted cells harboring the endogenously integrated sequence, so that, by addition of a selection agent (e.g., G418 or mycophenolic acid) such targeted cells have a growth or survival advantage over cells which do not have an integrated sequence.

It is preferable that selection for correctly targeted homologous recombinants also employ negative selection, so that cells bearing only non-homologous integration of the transgene are selected against. Typically, such negative selection techniques employ an expression cassette encoding the herpes simplex virus thymidine kinase gene (HSV tk) positioned in the transgene so that it integrates only by non-homologous recombination. Such positioning generally as accomplished by linking the HSV tk expression cassette (or other negative selection marker) distal to the recombinant homology regions so that double-crossover replacement recombination of the homology regions transfers the positive selection expression cassette to a chromosomal location but does not transfer the HSV tk gene (or other negative selection marker) to a chromosomal location. A nucleoside analog, gancyclovir, which is preferentially toxic to cells expressing HSV tk, can be used as the negative selection agent, as it selects for cells which do not have an integrated HSV tk expression marker. FIAU may also be used as a selective agent to select for cells lacking HSV tk.

Generally, targeting constructs of the invention include: (1) a positive selection marker flanked by two homology regions that are substantially identical to host cell endogenous BACE1 gene sequences, and (2) a distal negative selection marker. However, targeting constructs which include only a positive selection marker can also be used. Typically, a targeting construct will contain a positive selection marker, which includes a neo gene linked downstream (i.e., towards the carboxy-terminus of the encoded polypeptide in translational reading frame orientation) of a promoter such as the HSV tk promoter or the pgk promoter.

It is preferred that targeting constructs of the invention have homology regions that are highly homologous to the predetermined target endogenous DNA sequence(s), preferably isogenic (i.e., identical sequence). Isogenic or nearly isogenic sequences may be obtained by genomic cloning or high-fidelity PCR amplification of genomic DNA from the strain of non-human animals which are the source of the ES cells used in the gene targeting procedure.

For making transgenic non-human animals (which include homologously targeted non-human animals), embryonal stem cells (ES cells) are preferred. The embryonic stem cells described herein can be obtained and manipulated according to published procedures (Teratocarcinomas and Embryonic Stem Cells: A Practical Approach, E. J. Robertson, ed., IRL Press, Washington, D.C. (1987); Zjilstra et al., Nature 342:435-438 (1989); and Schwartzberg et al., Science 246:799-803 (1989), each of which is incorporated herein by reference). Murine ES cells, such as AB-1 line grown on mitotically inactive SNL76/7 cell feeder layers (McMahon and Bradley (1990) Cell 62: 1073) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the El 4 line (Hooper et al. (1987) Nature 326: 292-295), the D3 line (Doetschman et al. (1985) J. Embryol. Exp. Morph. 37: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal). The blastocysts containing the injected ES cells are allowed to develop in the uteri of pseudopregnant nonhuman females and are born as chimeric mice. The resultant transgenic mice are chimeric for cells having inactivated endogenous BACE1 loci and are backcrossed and screened for the presence of the correctly targeted transgene(s) by PCR or Southern blot analysis on tail biopsy DNA of offspring so as to identify transgenic mice heterozygous for the inactivated BACE1 locus. By performing the appropriate crosses, it is possible to produce a transgenic non-human animal homozygous for functionally disrupted BACE1 alleles. Such transgenic animals are substantially incapable of making an endogenous BACE gene product.

Non-human animals comprising transgenes which are heterozygous null or homozygous null for BACE1 can be used commercially as controls or standards in the development of AD therapeutics and diagnostics. For example, it is contemplated that the BACE-knockout organisms of the invention can be used as controls in screens for agents having the effect of lowering Aβ production and/or accumulation. Such agents can be developed as pharmaceuticals for treating abnormal APP processing and/or Alzheimer's disease, amongst other neurodegenerative conditions. Other uses include using cells (particularly neuronal cells) derived from the BACE1-knockout organisms for creating protein expression profiles between BACE1-knockout organisms and organisms of identical species having a phenotype associated with Alzheimer's Disease.

The effect of test agents on test animals, including transgenic animals, may be measured in various specimens from the test animals. In all cases, it will be necessary to obtain a control value which is characteristic of the level of production of APP and Aβ polypeptide and peptides in animals lacking a phenotype associated with AD. Accordingly, the transgenic animals of the invention (e.g., BACE1 knockout organisms) provide an ideal source of control organisms for studying AD as well as for screening the effects of agents on organisms having an AD-associated phenotype. Once such control level is determined, test compounds can be administered to additional test animals, where deviation from the average control value indicates that the test compound had an effect on the β-secretase activity in the animal. Test substances which are considered positive, i.e., likely to be beneficial in the treatment of Alzheimer's disease or other β-amyloid-related conditions, will be those which are able to reduce the level of ATF-βAPP production, preferably by at least 20%, more preferably by at least 50%, and most preferably by at least 80% or which display a phenotype substantially identical or superior to the phenotype of the BACE1-knockout organisms of the invention.

As used herein, “Alzheimer's Disease-associated phenotype” includes organisms having an a progressive formation of insoluble amyloid plaques and vascular deposits of the 4-kD amyloid β-peptide. In addition, the phenotype can result in organisms displaying impaired performance on memory learning tests and abnormal neuropathology in a cortico-limbic region of the brain.

The test agents can be any molecule, compound, or other substance which can be added to the cell culture or administered to the test animal without substantially interfering with cell or animal viability. Suitable test agents may be small molecules, biological polymers, such as polypeptides, polysaccharides, polynucleotides, and the like. The test compounds will typically be administered to transgenic animals at a dosage of from 1 ng/kg to 10 mg/kg, usually from 10 ug/kg to 1 mg/kg.

Test compounds which are able to inhibit secretion or animal production or generate a phenotype substantially identical to the BACE1-knockout organisms of the invention (e.g., having a reduce or negligible amount Aβ1-40, Aβ1-42, Aβ11-40, Aβ11-42 peptides) are considered as candidates for further determinations of the ability to block β-amyloid production in animals and humans. Inhibition of secretion or production indicates that cleavage of PAPP at the amino-terminus of βAP has likely been at least partly blocked, reducing the amount of a processing intermediate available for conversion to β-amyloid peptide.

The present invention further comprises pharmaceutical compositions incorporating a compound selected by the above-described method and including a pharmaceutically acceptable carrier. Such pharmaceutical compositions should contain a therapeutic or prophylactic amount of at least one compound identified by the method of the present invention. The pharmaceutically acceptable carrier can be any compatible, non-toxic substance suitable to deliver the compounds to an intended host. Sterile water, alcohol, fats, waxes, and inert solids may be used as the carrier. Pharmaceutically acceptable adjuvants, buffering agents, dispersing agents, and the like may also be incorporated into the pharmaceutical compositions. Preparation of pharmaceutical conditions incorporating active agents is well described in the medical and scientific literature. See, for example, Remington's Pharmaceutical Sciences, Mack Publishing Company, Easton, Pa., 16th Ed., 1982, the disclosure of which is incorporated herein by reference.

The pharmaceutical compositions just described are suitable for systemic administration to the host, including both parenteral, topical, and oral administration. The pharmaceutical compositions may be administered parenterally, e.g. subcutaneously, intramuscularly, or intravenously. Thus, the present invention provides compositions for administration to a host, where the compositions comprise a pharmaceutically acceptable solution of the identified compound in an acceptable carrier, as described above.

Transgenic organisms and/or effects of agents on organisms (e.g., organisms having a phenotype associated with AD) can be screened for presence of the transgene or changes in AD phenotypes in several ways. For example, brain APP protein and RNA expression can be detected and analyzed and the copy number and/or level of expression are determined using methods known to those of skill in the art. The transgenic animals or organisms displaying a phenotype associated with AD can also be observed for clinical changes. Examples of neurobehavioral disorders for evaluation are poor mating response, agitation, diminished exploratory behavior in a novel setting, inactivity, seizures and premature death.

For a particular strain, organism or transgene, sufficient copies of an APP gene and/or a sufficient level of expression of a coding sequence derived from a particular APP gene which will result in observable clinical and/or behavioral symptoms, together with a measurable biochemical change in relevant brain structures can be determined empirically. Various changes in phenotype are of interest. These changes may include progressive neurologic disease in the cortico-limbic areas of the brain expressed within a short period of the time from birth; increased levels of an APP gene or gene product above that of BACE1-knockout organisms and the development of a neurologic illness accompanied by premature death; gliosis and intracellular APP/Aβ accretions present in the hippocampus and cerebral cortex; progressive neurologic disease characterized by diminished exploratory/locomotor behavior, impaired performance on memory and learning tests, and diminished 2-deoxyglucose uptake/utilization and hypertrophic gliosis in the cortico-limbic regions of the brain. Such phenotypic characteristics or changes thereof can be used to identify agents which are of interested for further study in the treatment of AD. Such changes can be measurably compared to BACE1-knockout mice as a standard or control organism.

The animals can also be studied using a species appropriate neurobehavioral test. For example, studies of locomotor/exploratory behavior in mice is a standard means of assessing the neuropsychology (File and Wardill, (1975) Psychopharmacologia (Berl) 44:53-59; Loggi et al., (1991) Pharmacol. Biochem. Behav. 38:817-822). For example, for mice the “corner index” (CI) is used. This is a quick and simple neurobehavioral test to screen animals for evidence of brain pathology. The CI in transgenic mice which express mutant and wild-type APP is also measured and can be compared to similar behavior in BACE1-knockout mice as a control. A low CI correlates with high mutant APP copy numbers, premature death, and neuropathologic findings. The CI exhibits a dosage dependent relationship to APP copy number, which supports the validity of its use in assessing neurobehavioral signs in transgenic mice. The neuropathology of the animals also is evaluated. For rats, the Morris water maze test (described in Morris, (1984) J. Neurosci. Meth. 11:47), is used. A modified version of this test can be used with mice.

Brain regions known to be affected by the syndrome of interest are particularly reviewed for changes. When the disease of interest is Alzheimer's disease, the regions reviewed include the cortico-limbic region, including APP/Aβ excretions, gliosis, changes in glucose uptake and utilization and AP plaque formation. However, in strains of animals which are not long-lived, either naturally or when expressing high levels of APP, not all behavioral and/or pathological changes associated with a particular disease may be observed. As an example, transgenic FVB/N mice expressing high levels of APP tend not to develop detectable Aβ plaques, whereas longer lived C57B6/SJL F1 mice expressing identical transgenes do develop amyloid plaques which are readily detected with thioflavin S and Congo red. Immunologic studies of various brain regions also are used to detect transgene product. Comparing any of the foregoing with BACE1-knockout organisms can provide useful information in identifying novel therapeutic agents and diagnostics.

The transgenic organisms (e.g., BACE1 knockout organisms) of the invention can be used as controls for tester organisms for agents of interest, e.g. antioxidants such as Vitamin E or lazaroids, thought to confer protection against the development of AD. A test organism is treated with the agent of interest, and the neuropathology or behavioural pathology is compared to the BACE1-knockout organisms of the invention, wherein a neuropathology or behaviour in the test animal treated with the agent of interest that is substantially similar to or superior to that of the BACE1-knockout organisms is an indication of protection from AD. The indices used preferably are those which can be detected in a live animal, such as changes in performance on learning and memory tests. The effectiveness can be confirmed by effects on pathological changes when the animal dies or is sacrificed.

Careful characterization of the transgenic animals of the invention should lead to elucidation of the pathogenesis of progressive neurologic syndromes such as AD. The sequence of molecular events in BACE1 metabolism leading to disease can be studied. In addition, understanding the role and activity of BACE1 homologues including, for example, BACE2, are provided by the transgenic organisms of the invention. The animals also are useful for studying various proposed mechanisms of pathogenesis, including horizontal transmission of disease. Such knowledge would lead to better forms of treatment for neurologic disorders.

The following examples are provided as a guide for those skilled in the art, and are not to be construed as limiting the invention in any way. All products are used according to manufacturer's instructions, and experiments are conducted under standard conditions, unless otherwise specified.

### EXAMPLES

### Example 1

### Gene Targeting Vector and Embryonic Stem (ES) Cells.

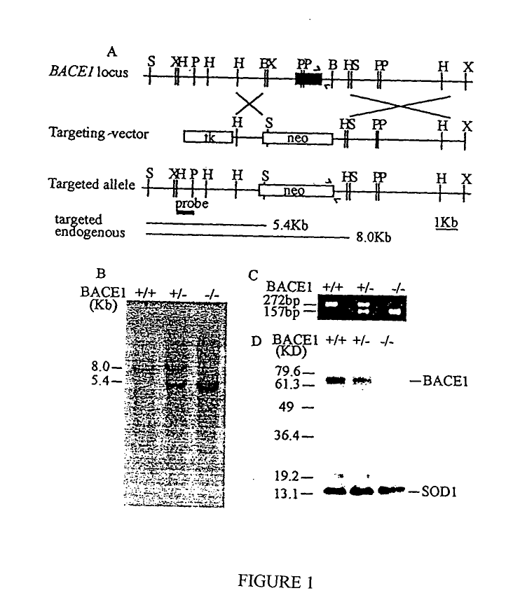

To examine the physiological roles of BACE1 and to determine whether BACE1 is the major P-secretase in neurons, mice with targeted inactivation of BACE1 alleles were developed. A homologous recombination strategy in embryonic stem (ES) cells was used to inactivate the mouse BACE1 gene. To target the BACE gene in ES cells, BACE genomic clones were isolated from a 129/Sv strain of mouse Lambda FIX II Library (Stratagene, Calif.) by using a partial mouse BACE cDNA containing the translation initiation codon as probe. In the BACE1 targeting vector, a 2.0-kb BamHI fragment containing the first coding exon which encode residues 1-87 (including the pro-peptide shown to be important for regulating BACE1 activity and flanking intronic sequences of the BACE1 gene was replaced with a neomycin-resistance gene (FIG. 1A) under the control of the PGK promoter. Introduction of a negative selection marker, the herpes simplex virus thymidine kinase gene, at the 5′ end of the construct allowed the use of the positive and negative selection scheme. The targeting vector was linearized at a unique NotI site before transfection into R1 ES cells, which were subjected to double selection. R1 ES cells were transfected with the linearized BACE1 targeting vector, and 2 clones (out of 112 screened) were targeted at the BACE1 locus. Clones were picked and expanded, and DNA was isolated from a portion of the cells and screened by Southern blot analysis. Targeted cells were expanded and injected into C57BL/6J blastocysts to produce highly chimeric male mice that transmitted the targeted BACE allele in the germline. BACE^{+/-} mice were intercrossed to obtain the BACE^{-/-} animals. BACE1-targeted ES cells were used to generate the BACE1^{-/-} mice. Genotype analyses of the BACE1^{-/-} mice were performed by DNA blotting (FIG. 1B) and PCR methods (FIG. 1C). Genotypes were determined by PCR amplification of tail or yolk sac DNA. The primer set (HC69: 5′AGGCAGCTTTGTGGAGATGGTG (SEQ ID NO:1); HC70: 5′CGGGAAATGGAAA GGCTACTCC (SEQ ID NO:2); and HC77: 5′TGGATGTGGAATGTGTGCGAG (SEQ ID NO:3)) was used to detect the endogenous and targeted BACE alleles. To confirm the targeting event led to inactivation of the BACE1 gene, protein immunoblotting analysis of brain extracts with a highly specific anti-BACE1 fusion protein antibody was performed. In BACE1^{+/-} mice, BACE1 accumulated to ˜50% the level of control littermate in brain, whereas the brain of BACE1^{-/-} mice showed no detectable level of BACE1 (FIG. 1D). Similar results were also observed using an antisera specific to the carboxyl-terminal 13 residues of BACE1 (data not shown). These results confirm the inactivation of BACE1.

### Example 2

### Antisera Preparation

BACE anti-peptide and anti-fusion protein antibodies were generated in rabbits. A synthesized peptide corresponding to the C-terminal 12 residues of mouse BACE coupled to KLH was used to make the anti-peptide antibody (Research Genetics, Huntsville, Ala.). To generate the HIS₆-BACE fusion protein, a DNA fragment corresponding to residues 46 to 163 of BACE was subcloned into pTrcHisA (Invitrogen, San Diego, Calif.). The HIS₆-BACE fusion protein purified by Talon Metal Affinity Resin (Clontech, Palo Alto, Calif.) chromatography was used as antigen for making the anti-fusion protein antibody (Covance Research Products Inc., Denver, Pa.).

### Example 3

### Generation of Human APP and BACE Recombinant Adenoviruses

A full-length human BACE cDNA was constructed from a near full-length clone isolated from a human fetal brain cDNA library (Origene Technologies Inc., MD) and a 5′ cDNA encoding the N-terminal 41 amino acids of BACE obtained by RT-PCR of total RNA from HEK293 cells. Recombinant adenoviruses expressing wild type/mutant human APP or BACE were produced by cloning the full-length wild type/mutant human APP or BACE cDNA, respectively, into the pAd-Track-CMV shuttle vector. Under the control of distinct CMV promoters, this plasmid expresses the human APP or BACE, and in parallel, green fluorescent protein (GFP). The construct was integrated into the adenoviral backbone vector, pAd-Easy-1, by homologous recombination in Escherichia coli strain BJ5183. The adenoviral construct was then cleaved with PacI and transfected in a packaging cell line (HEK 293 cells). The titer of the viral stocks was estimated based on the density of GFP-expressing cells.

### Example 4

### Primary Cortical Cultures and Metabolic Labeling

Cortical neuronal cultures were established from brains of embryonic day 16.5 fetal mice. The dissected brain cortexes were suspended in HBSS supplemented with 0.25% trypsin and 0.01% DNaseI and incubated at 37° C. for 10 min. The tissues were then transferred to Dulbecco's modified Eagle's medium (DMEM) supplemented with 10% fetal bovine serum and dissociated by repeated trituration. The dispersed cells were collected by centrifugation and plated at ˜1×10⁶ cells/well on 6-well cell culture plates (coated with poly-D-lysine) in B27/Neurobasal media (GIBCO/BRL, Gaithersburg, Md.). Neurons were allowed to mature for 4-7 days in culture before they were used for experiments. Primary neuronal cells cultured for 4 to 7 days were infected with 5×10⁶ plaque-forming units of adenovirus expressing human APP for 4 days in serum-free medium. For metabolic labeling, neuronal cells were pre-incubated for 30 min in methionine-free DMEM with 1% dialyzed bovine serum and then labeled with 700 μCi/ml of ³⁵S-methionine in methionine-free medium for 5 hr. For pulse-chase labeling, cells were pulsed for 45 min with methionine-free DMEM containing 1 mCi/ml ³⁵S-methionine. Cells were then chased by washing and incubating in DMEM containing 1% dialyzed fetal bovine serum and 1 mM L-methionine at varying intervals, before the cells were lysed in immunoprecipitation buffer containing detergents and a protease inhibitor cocktail. After metabolic labeling, culture medium and cell extracts were immunoprecipitated and immunoprecipitates were fractionated on either 4%-20% Tris-glycine or 16% Tris-tricine SDS-PAGE. Gels were dried, exposed, and radioactive bands were quantified by phosphorimaging analysis.

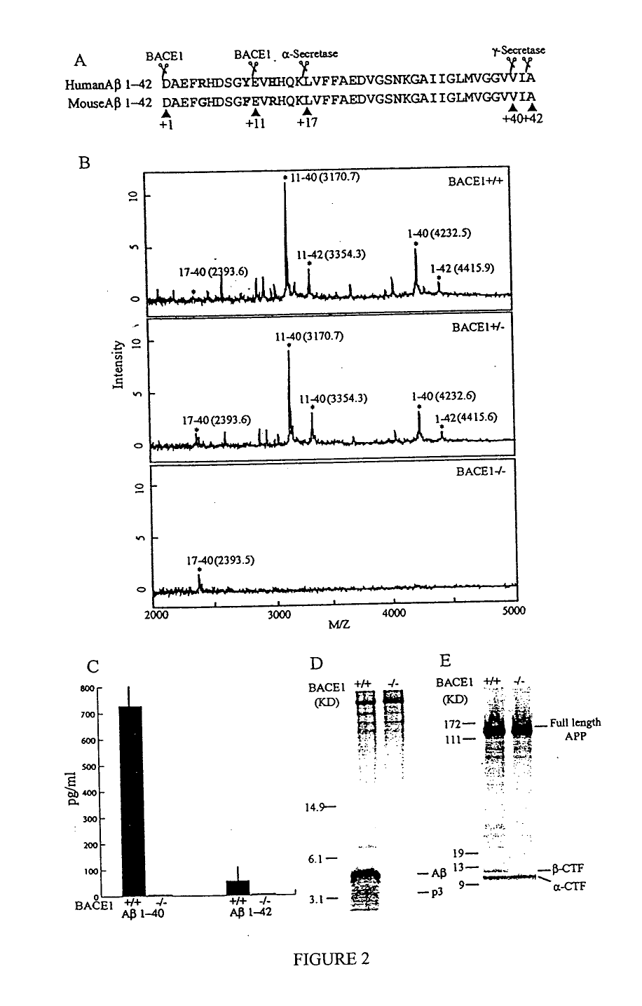

To examine the effect of the absence of BACE1 on secretion of Aβ peptides from neurons, primary cortical cultures from control, BACE1^{+/-} and BACE1^{-/-} embryos were derived from day 16.5 post coitum. The growth rate and morphology of the BACE1^{-/-} cultures were identical to those of the BACE1^{+/-} or control. Immunoprecipitation-mass spectrometry (IP-MS) analysis of conditioned culture media from control neurons after 5 days in culture using an antisera (4G8) specific to epitopes between residues 17-28 of Aβ revealed two prominent Aβ species with mass values of 3171 and 4233 corresponding to mouse Aβ11-40 and Aβ1-40 respectively, in addition to several minor species including Aβ311-42 and Aβ1-42 (FIG. 2B). While these Aβ species are similarly observed in conditioned culture media from BACE1^{+/-} neurons, secretion of these Aβ species is abolished from BACE1^{-/-} neurons except for the Aβ17-40 (p3) fragment (FIG. 2B). These data establish that BACE1 is the major β-secretase required for cleavages of βAPP at the +1 and +11 sites of Aβ peptide in embryonic cortical neurons. Because a primary cleavage site for BACE2 is at +19/+20 of Aβ and no Aβ20-40/42 or Aβ21-40/42 was detected, it was inferred therefore that BACE2 plays little role in the cleavage of APP in neurons.

To confirm the unique role of BACE1 in neurons, the processing of APP in control and BACE1^{-/-} neuronal cultures following infection with a recombinant adenovirus expressing a humanized APP cDNA (a murine APP cDNA in which the Aβ1-42 region corresponds to the human Aβ1-42) bearing the Swedish variant (APPswe) was examined. Quantitative sandwich ELISA analyses of conditioned media from BACE1^{+/+} cultures expressing APPswe showed high levels of Aβ1-40 and Aβ1-42 while undetectable levels of Aβ1-40 and Aβ1-42 were observed from media of BACE^{-/-} cultures expressing APPswe (FIG. 2C). Metabolic labeling of control and BACE1^{-/-} cortical neurons with ³⁵S-methionine for 5 hours and immunoprecipitation analysis using 4G8 antisera showed the presence of a major band (˜4 kD) corresponding to Aβ and a minor band (˜3.2 kD) corresponding to p3 in control culture (FIG. 2D), but, although p3 is readily secreted, no Aβ accumulated in conditioned media from BACE1^{-/-} cultures expressing APPswe (FIG. 2D). Moreover, immunoprecipitation analysis using CT15, an antibody specific for the carboxyl-terminal 15 residues of APP¹², revealed in BACE1^{-/-} detergent lysates the accumulation of full length APP as well as APP α-CTF (FIG. 2E); however, this approach failed to detect APP β-CTF in the lysates, which are in control lysates (FIG. 2E). Taken together, these results confirm that BACE1 is the primary β-secretase in cortical neurons and infer that BACE2 does not play a significant role in the processing of APP in neuronal cultures.

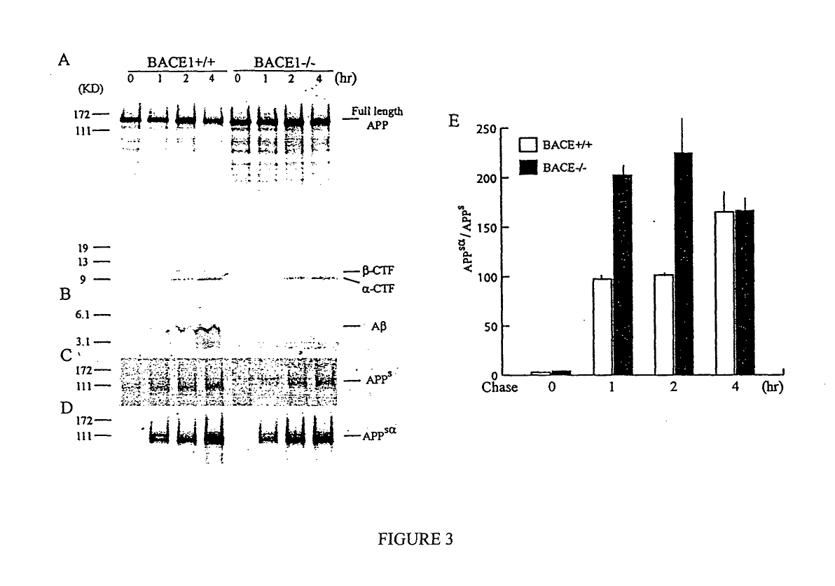

Since β- and α-secretases compete for the same substrate, we anticipated that in the absence of BACE, APP derivatives produced by the action of α-secretase might be increased. To determine whether the rate of secretion of α-secretase derived APP soluble ectodomain (APPsα) is altered, the processing of APPswe in BACE1^{+/+} and BACE1^{-/-} cortical neurons was examined. Pulse-chase studies revealed that there is an increase in the rate of secretion of APPsα in BACE1^{-/-} neurons as compared to controls (FIGS. 3C-E). Furthermore, no accumulation of either β-CTF or AP in the BACE1^{-/-} neuronal cultures was detected (FIGS. 3A, B). These results establish that BACE1 competes with α-secretase in APP processing and further confirm the view that BACE1 is the major P-secretase in neurons.

### Example 5

### Mass Spectrometric Analysis

The β-amyloid peptides were captured with 4G8 monoclonal antibody (Senetek, Napa, Calif.) by immunoprecipitation from conditioned media of cultured neurons. After final wash, the immunoprecipitates were rinsed twice with 5 mM HEPES buffer (pH 7.0). 1 μl sample was spotted on NP-1 series ProteinChip array and analyzed by surface-enhanced laser desorption/ionization time of flight MS (Ciphergen Biosystems, Palo Alto, Calif.) in the presence of CHCA matrix solution (Ciphergen Biosystems). External standards were used for calibration.

### Example 6

### Determination of Aβ1-42/43 and Aβ1-40 Levels

Two-site ELISAs that specifically detect the C-terminus of AP were performed to measure Aβ levels as suggested by the manufacturer (Biosource International, Camarillo, Calif.). Culture media of neuronal cells infected with adenovirus expressing human APP were collected and analyzed using the quantitative sandwich ELISA to determine both AP 1-42 and Aβ1-40 levels.

To confirm that BACE1 cleaves APP at both the +1 and +11 sites of Aβ, neuronal cultures infected with adenovirus expressing either humanized wild type APP (hAPPwt) or its variants (hAPPswe or hAPP717) were examined and the secretion of Aβ peptides from conditioned media as well as the accumulation of both +1 and +11 derived β-CTFs from cell lysates measured. As expected, IP-MS analysis of conditioned media using 4G8 antibody show that the human and murine Aβ1-40 and Aβ1-42 are secreted, however, the human Aβ11-40 peptide is not secreted into culture media from murine primary neurons infected with adenovirus expressing hAPPwt, although the murine Aβ11-40 is readily detected. Similar results are also observed with murine neurons infected with adenovirus expressing hAPPswe or hAPP717. This apparent discrepancy raised the possibility that the cleavage site at +11 of AP is species-specific, i.e., human or murine BACE1 cleaves respectively, human or murine APP at +11 site of Aβ whereas no species selectivity occurs at the +1 site. To test this possibility, the processing of human APP by co-infecting murine neuronal cultures with adenovirus expressing both human BACE1 and hAPPwt or its variants was examined. IP-MS analysis of conditioned media using 4G8 antibody now revealed the secretion of human Aβ11-40 peptide in addition to the murine Aβ11-40 peptide from murine neurons co-expressing human BACE1 and hAPPwt. The human Aβ11-40 peptides are also secreted by primary neurons co-expressing human BACE1 and hAPPsw or hAPP717.

In addition, since human BACE1 cleaves human APP at the +11 site of Aβ the +11 derived P-CTF was examined to determine whether it accumulated in lysates of neurons co-expressing human BACE1 and human APPwt or its variants. As expected, while α-CTFs are readily immunoprecipated using the CT15 antibody from control, hAPPwt, hAPPswe or hAPP717 lysates, the +1 derived p-CTF is observed only in the hAPPswe lysate. However, when neurons co-expressing human BACE1 and hAPPwt or hAPPswe or hAPP717 there is secreted a peptide corresponding to the +11 derived β-CTF (+11-CTF) in addition to the +1 derived β-CTF. Taken together, these results support the view that the cleavage site at +11 of Aβ is species-specific. To begin to access the determinants that govern this selectivity, the amino acid sequences of Aβ between humans and mice were compared; there is a sequence divergence around the +11 site whereas there is absolute conservation at the +1 site of Aβ (see FIG. 2A). Mutagenesis studies will allow determination of the amino acid residue(s) that confer species specificity at the +11 site of Aβ. Although Aβ11-40/42 peptides has been previously observed in neuronal cultures as well as in the brains of cases of AD, the roles of these peptides in the pathogenesis of AD was not understood. Aβ beginning at +11 is a major species in rodents in vivo and this peptide is more fibrillogenic and neurotoxic than full length Aβ in vitro. Because the finding that the +11 site is a major cleavage site for BACE1, the involvement of Aβ11-40/42 in pathogenesis of AD is important. Aβ11-40/42 plays a critical role in AD, thus antibodies specific to AP 11-40/42 would prove useful for diagnoses of sporadic AD. The demonstration that the cleavage at +11 is species-specific would infer that the published mutant human APP transgenic models 17-19 would not be expected to secrete the human Aβ11-40/42 (because murine BACE1 does not cleave at the +11 site) and transgenic mice overexpressing either murine wild type APP or its variants may be instructive in clarifying the pathogenic roles of Aβ1-40/42.

The secretion of Aβ peptides (Aβ1-40/42 as well as Aβ11-40/42) from neurons is abolished in cultures of BACE1-deficient embryonic cortical neurons derived from BACE1-knockout mice. Moreover, while the intracellular β-carboxy terminal fragments of βAPP (β-CTFs) and the corresponding APPsβ fragments are not generated in BACE^{-/-} neurons, the rate of APPsα secretion is increased in BACE^{-/-} neurons as compared to controls. These results establish that BACE1 is the principal neuronal protease required to cleave β-amyloid precursor protein (APP) at +1 and +11 sites of Aβ that generate N-termini of Aβ. In addition, the invention provides for the first time, that while both human and murine BACE1 are capable of cleaving either human or murine βAPP at the +1 site of Aβ, the cleavage at the +11 site is species-specific. Taken together, these results have important implications for the development of novel therapeutic strategies in Alzheimer's disease.

While both β- and γ-secretase activities represent therapeutic targets for the development of novel protease inhibitors for AD, the discovery of BACE1 and BACE2 now provides the opportunity to determine whether these aspartic proteases are indeed high priority targets. The demonstration that BACE1 is the major β-secretase in neurons provides excellent rationale for focusing on the design of novel therapeutics to inhibit BACE1 activity in brain as well as using Aβ11-40//42 as novel tools for diagnosing AD. The transgenic organisms of the invention allow for the identification of other important substrates for BACE1 and the evaluation of BACE1 knockout. This information will have significant impact in the design of specific drugs to inhibit BACE1 in the central nervous system. To illustrate this principle, it is instructive to consider the emerging view that the presenilins (PS1 and PS2), which when mutated cause familial AD and which are important for the intramembraneous proteolysis of several proteins, including APP and Notch1, may be the putative γ-secretase. Presenilins are involved in the proteolytic processing of Notch1 and they are critical for Notch1 functions. PS1 null mice, which die before or at birth, have a developmental defect in patterning of somites; a phenotype resembling that observed in the Notch1 null mice. Recent demonstrations that PS1 co-fractionates with γ-secretase activity, that transition-state analogue inhibitors of γ-secretase can covalently label PS, and that two transmembrane aspartates are required for γ-secretase activity provide support for the view that PS1/2 may possess γ-secretase activity or is a co-factor intimately associated with γ-secretase cleavages. Alternatively, PS1/2 may play a role in trafficking of APP or other molecules. Consistent with the idea that γ-secretase activity is subserved by a multi-subunit catalytic complex is the recent identification of the type 1 transmembrane protein, nicastrin, which interacts with presenilins that are known to modulate both γ-secretase activity and Notch1 function. Thus, the design of therapeutics that inhibit γ-secretase and thus influence Notch1 processing could have in the adult, impact on some cell populations (hematopoetic cell) that utilize Notch1 signaling for cell fate decision. In this case, it would be necessary to try to develop highly selective inhibitors that act principally on γ-secretase activities that cleave APP and have less inhibiting potency on Notch1 cleavage. The demonstration that BACE1 null mice are viable allows for the development of inhibitors that are brain penetrate (i.e., can cross the blood brain barrier), bind to the active sites (extracellular) of BACE1 to ameliorate β-amyloid deposition, and are without profound adverse effects. BACE1 null mice are valuable for testing whether the β-amyloid burden can be reduced in mutant APP transgenic models lacking BACE1. Such an outcome would greatly encourage investigators to design novel drugs to inhibit BACE1 activity. The recent report documenting the crystal structure of the protease domain of BACE1 associated with an eight-residue inhibitor provide valuable information towards the development of specific drugs to inhibit BACE1 activity. These compounds can be tested in transgenic mice to determine whether they ameliorate Aβ deposition. If so, these therapeutic can be brought rapidly into clinical trials.

Although the invention has been described with reference to the certain embodiments, it should be understood that various modifications can be made without departing from the spirit of the invention. Accordingly, the invention is limited only by the following claims.

What is claimed:

1. A method for modulating the production of Aβ11-40/42 peptide fragments comprising contacting a sample or cell containing a beta-site APP-cleaving enzyme 1 (BACE1) and an amyloid precursor protein (APP) with a BACE1-modulating agent such that production of Aβ11-40/42 is modulated.

2. The method of claim 1, wherein the modulation is inhibition of Aβ11-40/42 peptide formation.

3. The method of claim 1, wherein the contacting is in vivo.

4. The method of claim 1, wherein the contacting is in vitro.

5. The method of claim 1 wherein the BACE1-modulating agent is an anti-BACE1 antibody or a BACE1 antisense molecule.

6. A method for identifying a compound which inhibits beta-site APP-cleaving enzyme 1 (BACE1) expression or activity comprising:
a) incubating components comprising the compound, BACE1 polynucleotide or polypeptide, and an amyloid precursor protein (APP) under conditions sufficient to allow the components to interact; and
b) measuring the production of a BACE1 specific enzymatic product.

7. The method of claim 6, wherein the compound is a peptide.

8. The method of claim 6, wherein the compound is a small molecule inhibitor.

9. The method of claim 6, wherein the BACE1 polynucleotide or polypeptide is expressed in a cell.

10. The method of claim 6, wherein the BACE1 specific enzymatic product includes a sequence of Aβ11-40/42.

11. A compound identified by the method of claim 6.

12. The compound of claim 11, in a pharmaceutically acceptable carrier.

13. A method for diagnosing a subject having or at risk of having an Aβ11-40/42 peptide accumulation disease, the method comprising:
measuring the amount of beta-site APP-cleaving enzyme 1 (BACE1) in a biological sample from the subject;
comparing the amount BACE1 with a normal standard value of BACE1, wherein a difference between the measured amount and the normal sample or standard value provides an indication of the diagnosis of Aβ11-40/42.

14. The method of claim 13, wherein the biological sample is blood, serum, cerebrospinal fluid or central nervous system (CNS) tissue.

15. The method of claim 13, wherein the difference is an increase in BACE1.

16. The method of claim 13, wherein the amount BACE1 is measured by detecting the amount of a polynucleotide encoding BACE1.

17. The method of claim 16, wherein the polynucleotide is mRNA.

18. The method of claim 17, wherein the mRNA is detected by PCR.

19. The method of claim 13, wherein the amount of BACE1 is detected by contacting the sample with an agent that specifically binds to a BACE1 polypeptide.

20. The method of claim 19, wherein the agent is an antibody.

21. The method of claim 20, wherein the antibody is a monoclonal antibody.

22. The method of claim 20, wherein the antibody is a polyclonal antibody.

23. The method of claim 19, wherein the Aβ11-40/42 accumulation disease is Alzheimer's Disease.

24. The method of claim 13, further comprising detecting the level of an APP fragment, wherein an increase in the presence of the fragment is indicative of Alzheimer's Disease.

25. The method of claim 24, wherein the APP fragment is a Aβ1-40, Aβ1-42, Aβ11-40, or Aβ11-42 fragment.

26. The method of claim 25, wherein the fragments are detected by contacting the sample with an agent the specifically binds to Aβ1-40, Aβ11-42, Aβ11-40, or Aβ11-42 fragment.

27. The method of claim 26, wherein the agent is an antibody.

28. The method of claim 20 or 27, wherein the antibody is detectably labeled.

29. The method of claim 28, wherein the detectable label is selected from the group consisting of a radioisotope, a bioluminescent compound, a chemiluminescent compound, a fluorescent compound, a metal chelate, and an enzyme.

30. A method for diagnosing a subject having or at risk of having Alzheimer's Disease, the method comprising:
measuring Aβ11-40/42 in a biological sample from the subject;
comparing the amount of Aβ11-40/42 with a normal sample or standard value of Aβ11-40/42, wherein a difference between the amount in the normal sample or standard value is indicative of a subject having or at risk of having Alzheimer's disease.

31. The method of claim 30, wherein the biological sample is cerebrospinal fluid, central nervous system (CNS) tissue, serum or blood.

32. The method of claim 30, wherein the difference is an increase in Aβ11-40/42 and the increase is indicative of a disposition for Alzheimer's disease.

33. The method of claim 30, wherein the difference is a decrease in Aβ1-40/42.

34. The method of claim 30, wherein the amount of Aβ11-40/42 is detected by contacting the sample with an agent that specifically binds to Aβ11-40/42.

35. The method of claim 34, wherein the agent is an antibody.

36. The method of claim 35, wherein the antibody is a monoclonal antibody.

37. The method of claim 35, wherein the antibody is a polyclonal antibody.

38. The method of claim 34, wherein the agent is an antibody fragment.

39. The method of claim 30, further comprising detecting the level of a BACE1 polypeptide or polynucleotide, wherein an increase in the level of BACE1 is indicative of Alzheimer's Disease.

40. The method of claim 35, wherein the antibody is detectably labeled.

41. The method of claim 40, wherein the detectable label is selected from the group consisting of a radioisotope, a bioluminescent compound, a chemiluminescent compound, a fluorescent compound, a metal chelate, and an enzyme.

42. A transgenic non-human animal having a transgene disrupting expression of BACE1, chromsomally integrated into the germ cells of the animal, and have a phenotype of reduced Aβ peptide as compared with a wild-type animal.

43. The transgenic non-human animal of claim 42, wherein the animal is selected from the group of species consisting of avian, bovine, ovine, piscine, murine, and porcine.

44. The transgenic non-human animal of claim 42, wherein the animal is heterozygous or homozygous for the disruption.

45. The transgenic non-human animal of claim 42, wherein the transgene comprises a BACE1 antisense polynucleotide.

46. A method for producing a transgenic non-human animal having a phenotype characterized by reduced expression of BACE1 polypeptide, the method comprising:
(a) introducing at least one transgene into a zygote of an animal, the transgene(s) comprising a DNA construct encoding a selectable marker,
(b) transplanting the zygote into a pseudopregnant animal,
(c) allowing the zygote to develop to term, and
(d) identifying at least one transgenic offspring whose genome comprises a disruption of the endogenous BACE1 polynucleotide sequence by the transgene.

47. The method of claim 46, wherein the introducing of the transgene into the embryo is by introducing an embryonic stem cell containing the transgene into the embryo.

48. The method of claim 46, wherein the transgenic non-human animal is heterozygous or homozygous for the disruption.

49. The method of claim 46, wherein the introducing of the transgene into the embryo is by infecting the embryo with a retrovirus containing the transgene.

50. A method for identifying an agent that modulates the expression or activity of BACE1, said method comprising:
administering an agent to be tested to an organism; and
comparing the phenotype of the organism contacted with the agent with that of a BACE1-knockout organism not contacted with the agent, whereby a phenotype substantially equal to the BACE1-knockout organism is indicative of an agent that modulates BACE1 expression or activity.

51. The method of claim 50, wherein the organism is a transgenic organism.

52. The method of claim 51, wherein the transgenic organism is transgenic for overexpression of BACE1; APP expression; Aβ1-40, Aβ1-42, Aβ11-40, Aβ11-42 expression; or a combination thereof.

53. The method of claim 50, wherein the expression of BACE1 is detected by measuring the amount of BACE1 polynucleotide in the organism.

54. The method of claim 53, wherein the BACE1 polynucleotide is RNA or DNA.

55. The method of claim 54, wherein the RNA is mRNA.

56. The method of claim 50, wherein the activity of BACE1 is detected by measuring BACE1 cleavage of APP.

57. The method of claim 50, wherein the phenotype of the organism is associated with Alzheimer's Disease.

58. The method of claim 57, wherein the Alzheimer's-associated phenotype is characterized as having a phenotype of impaired performance on memory learning tests and abnormal neuropathology in a cortico-limbic region of the brain.

59. A method for screening for an agent, which ameliorates symptoms of Alzheimer's disease, said method comprising:
comparing an effect of an agent on an organism contacted with the agent with that of a BACE1-knockout organism not contacted with the agent, wherein the organism has a phenotype associated with Alzheimer's Disease and wherein an agent which ameliorates said phenotype is identified by having a substantially equal or superior phenotype of the organism in comparison with the BACE1-knockout organism.

60. The method of claim 59, wherein the phenotype of the organism is characterized as having a phenotype of impaired performance on memory learning tests and abnormal neuropathology in a cortico-limbic region of the brain.

61. The method of claim 59, wherein the organism is a transgenic organism.

62. The method of claim 59, wherein the phenotype is measured by assessing an organism's performance on memory and learning tests.

63. The method of claim 59, wherein the phenotype is measured by assessing the neuropathology in a cortico-limbic region of the brain.

64. A method for screening for an agent, which ameliorates symptoms of Alzheimer's disease, said method comprising:
comparing an effect of an agent on a transgenic organism contacted with the agent with that of a BACE1-knockout organism not contacted with the agent, wherein the transgenic organism is characterized as having a phenotype of impaired performance on memory learning tests or abnormal neuropathology in a cortico-limbic region of the brain and the BACE1-knockout organism has a phenotype of reduced expression of BACE1, wherein the impaired performance and the abnormal neuropathology are in compared with the BACE1-knockout organism, whereby an agent which ameliorates the symptoms is identified by substantially equal or superior performance of the transgenic organism as compared with the BACE1-knockout organism on the memory and learning tests.

65. A kit useful for the detection of an Aβ11-40/42 accumulation disorder comprising carrier means containing therein one or more containers wherein a first container contains a nucleic acid probe that hybridizes to a nucleic acid sequence BACE1 or an antibody probe specific for BACE1 or Aβ11-40/42.

66. The kit of claim 65, wherein the probe is detectably labeled.

67. The kit of claim 65, wherein the label is selected from the group consisting of radioisotope, a bioluminescent compound, a chemiluminescent compound, a fluorescent compound, a metal chelate, and an enzyme.

68. A method for predicting the therapeutic effectiveness of a compound for treating Alzheimer's disease in a subject comprising:
measuring the accumulation of AB 11-40/42 peptide fragments in the subject or the level of BACE1 polynucleotide or polypeptide before and after treatment with the compound, wherein a decrease in accumulation of peptide fragments or a decrease in the level of BACE1 polynucleotide or polypeptide after treatment is indicative of a compound that is effective in treating the disease.

69. A method for monitoring the progression of Alzheimer's disease comprising: measuring the accumulation of AB 11-40/42 peptide fragments in the subject or the level of BACE1 polynucleotide or polypeptide at a first time point and a second time point, thereby monitoring the progression of the disease.
